(19) United States
(12) Patent Application Publication
(10) Pub. No.: US 2025/0371673 A1
(43) Pub. Date: Dec. 4, 2025

(54) SYSTEMS AND METHODS FOR DENOISING IMAGES RENDERED FROM SCAN DATA ACQUIRED BY COMPUTED TOMOGRAPHY

(71) Applicant: Epica International, Inc., San Clemente, CA (US)

(72) Inventors: Elena Denisova, Florence (IT); Leonardo Bocchi, Florence (IT)

(21) Appl. No.: 19/214,994

(22) Filed: May 21, 2025

Related U.S. Application Data
(60) Provisional application No. 63/652,536, filed on May 28, 2024.
(60) Provisional application No. 63/676,222, filed on Jul. 26, 2024.

(51) Int. Cl. G06T 5/50; G06T 1/20; G06T 5/70; G06T 15/06; G16H 30/40

(52) U.S. Cl. CPC G06T 5/50; G06T 1/20; G06T 5/70; G06T 15/06; G16H 30/40; G06T 2207/20221

(57) ABSTRACT

Denoising images rendered from scan data acquired by computed tomography (CT), including receiving CT scan data and generating a grid of pixels, for a first pixel channel, based at least in part on the CT scan data, each pixel having an associated radiance value. Methods include iteratively tracing a plurality of rays originating at a camera position based at least in part on radiance values of intersected pixels to produce a Monte Carlo estimate image and applying a denoising algorithm to the Monte Carlo estimate image to produce a denoised image. Methods further include determining one or more weights based at least in part on the Monte Carlo estimate image and the denoised image. Methods further include blending the Monte Carlo estimate image and the denoised image based at least in part on said one or more weights to produce a rendered image.

### CROSS REFERENCE TO RELATED APPLICATIONS

The present application claims priority to U.S. Provisional Patent Application No. 63/652,536, filed May 28, 2024 and U.S. Provisional Patent Application No. 63/676,222, filed Jul. 26, 2024, each of which is hereby incorporated by reference in its entirety.

### BACKGROUND

### Technical Field

The present disclosure generally relates to systems and methods for denoising images rendered from scan data acquired by computed tomography (CT) and, in particular, for performing converging algorithm-agnostic denoising for Monte Carlo Rendering.

### Description of the Related Art

Highly accurate rendering, e.g., 3D rendering, of biomedical scan data has proven useful for faster comprehension of traumas in areas of high anatomic complexity, for surgical planning, simulation, and training. Also, it improves communication with patients, as they will understand a diagnosis more easily if it is rendered clearly in 3D. Therefore, most existing biomedical imaging systems provide different 3D rendering modalities for volume visualization.

Monte Carlo Path Tracing (MCPT) is a rendering technique capable of generating images that closely resemble photographs when accurately modeling both lighting and material properties. However, the inherently stochastic nature of Monte Carlo simulations inevitably introduces noise, particularly in initial iterations and areas with insufficient lighting, which stands as a primary drawback of this method. As scenes and lighting configurations grow more complex, the number of iterations required to produce a clear, noise-free image increases accordingly. Denoising algorithms are used to reduce noise in images. Noise, in this context, refers to random variations of brightness or color information in images that can be caused by electronic noise in the capture sensor of the digital camera, or due to less light or higher temperature, etc. Conventional denoising algorithms often struggle to differentiate high-frequency elements within rendered images from noise, leading to unintended over-blurring, even at very high sample counts. This loss of fine detail is evident in the spectral analysis of FIG. 2, where the lower amplitude of the higher frequencies indicates a suppression of these components within the denoised image.

Denoising images rendered using Monte Carlo Path Tracing (MCPT) is a challenge in computer graphics which is typically addressed in post-processing phases. Some conventional techniques, involving blending, require denoising at every MCPT iteration, which reduces the efficiency and output fidelity of the rendering process. Also, denoising can compromise output fidelity even if done at higher sample counts. Ensuring that the denoised image accurately converges to the reference is important, particularly in fields like biomedical rendering, where 3D representation has demonstrated its utility in quickly comprehending traumas in anatomically complex areas, aiding in surgical planning, simulation, and training. Conventional approaches which employ noise reduction as a post-processing step on the partially converged image can reduce the time needed to generate an acceptable result, but at the expense of accuracy.

Other conventional methods rely on deep-learning techniques. Specifically, recent studies advocate for blending denoised outputs with noisy inputs to enhance results and ensure convergence to the ground truth. However, because these approaches rely on neural networks trained on denoised images, re-training is necessary whenever the denoising algorithm changes.

### SUMMARY

Disclosed embodiments allow for integrating denoising directly into progressive rendering. These embodiments not only blend denoised outputs with noisy inputs to enhance visual quality but also guide adaptive sampling through variance estimation of denoised outputs. We introduce a method that predicts the blending weight of denoised images directly during progressive rendering. Our technique adjusts the blending weight for each pixel based on error estimates, effectively “skipping” certain iterations of Monte Carlo Path Tracing (MCPT) and mimicking adaptive sampling a posteriori. Among the advantages of our approach is that it provides an analytical method that ensures the blended output converges accurately to the reference image.

Disclosed embodiments provide an analytical solution for blending unbiased (e.g., hypothetically noisy Monte Carlo estimate) and biased (e.g., denoised) images, independent of the denoising algorithm employed, whether traditional or deep learning-based. Disclosed methods accommodate both progressive denoising, applied iteratively after each MC iteration to refine accumulated estimates, and progressive rendering, where denoising ceases after a predetermined number of iterations. In embodiments, disclosed methods can be used as a final step in the MCPT algorithm, helping to reconstruct details potentially lost during denoising when the algorithm converges. In contrast to real-time rendering with minimal ray budgets, disclosed methods perform progressive rendering, which is useful in generating photorealistic images. Methods disclosed herein accommodate both progressive denoising, occurring after each iteration, and progressive rendering, where denoising ceases after a predetermined number of iterations. In contrast to conventional approaches, the disclosed methods do not rely on deep-learning techniques and therefore does not require re-training when the denoising algorithm changes. Moreover, the disclosed approaches eliminate the need for denoising at every iteration. Instead, they allow for the blending of images acquired at varying sample counts, thereby enhancing computational efficiency and simultaneously improving the visual quality.

In embodiments, we compute blending weights for each pixel channel using both the Monte Carlo (MC) estimate and denoised images. These weights correspond to effective or “predicted” iteration numbers, e.g., numbers corresponding to the samples per pixel during MC simulations. As in adaptive sampling a posteriori, we use the Stein Unbiased Risk Estimate (SURE) for estimating the mean squared error (MSE) of denoised images against the reference. Such approaches rely on the input radiance values adhering to a normal distribution for SURE's accuracy.

We illustrate our approach using the Mitsuba framework for MCPT rendering, employing two pre-trained denoising models: Intel® Open Image Denoise (OIDN) with albedo and normal auxiliary features, and RT, a denoiser based on OIDN but operated on albedo, normal, and input mean variances. Our results are comparable with state-of-the-art deep-learning progressive denoising methods and even surpass them in some cases (see, e.g., FIG. 1).

We demonstrate the retrospective feasibility of the disclosed methods and describe their application in real-time scenarios, highlighting their ability to improve both rendering efficiency and quality. Additionally, our approach can be effectively integrated with adaptive sampling techniques, wherein weights assigned to each pixel guide the sampling process.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1 (a)-(f) are examples of images generated using the Mitsuba framework for MCPT rendering, employing two pre-trained denoising models: Intel® Open Image Denoise (OIDN) with albedo and normal auxiliary features, and RT, a denoiser based on OIDN but operated on albedo, normal, and input mean variances, wherein FIG. 1(a) is a 32k spp reference image, FIG. 1(b) illustrates excessive smoothing observed in denoising operations, particularly at relatively low sample per pixel (spp) counts, FIG. 1(c) illustrates that this phenomenon may occur even when using high-quality denoising algorithms such as OIDN with albedo and normal auxiliary features, FIG. 1(d) shows results achieved with learning-based progressive denoising (PD), FIG. 1(e) shows that the disclosed methods yield results comparable to, or exceeding, those achieved with learning-based PD, and FIG. 1(f) is an imaging serving as a reference crop.

FIG. 2 shows plots of a spectral analysis of the crown scene (see FIG. 1(a)) at 32k sample counts (spp) for the entire frequency range (left) and focused on the higher frequencies (right). The curves represent the filtered mean frequency amplitude calculated from the magnitude of the Fast Fourier Transform applied to the MC estimate and its denoised counterpart produced by Open Image Denoise (OIDN).

FIG. 3 is a plot of the mean variance of images generated with Mitsuba (right) in logarithmic scale, displaying the mean variance curves in logarithmic scale (left) for 10 different scenes generated with the Mitsuba framework for MCPT rendering, including the sanmiguel outdoor scene (middle of bottom row).

FIG. 4 shows three plots of an example of the mean variance of a single pixel per channel (Red, Green, and Blue channels, respectively).

FIG. 5 is a plot (left) of the mean squared error (MSE) of the Monte Carlo estimate, its denoised counterpart, and the “easy blending” solution for the sanmiguel outdoor scene of FIG. 3, and a crop of the scene (right) showing a comparison between (clockwise from upper left) 128 samples per pixel (spp) Monte Carlo estimate, OIDN, “easy blending,” and a 32k spp reference image.

FIGS. 6(a)-(d) present the mean variance of a cropped version of the sanmiguel scene of FIG. 3 at a sample count of four, wherein FIG. 6(a) shows a Monte Carlo estimate, FIG. 6(b) shows the mean variance, FIG. 6(c) shows the mean variance filtered by Gaussian blur, FIG. 6(d) depicts the mean variance denoised by OIDN, and the plots represent MSE of the slope (bottom left) and the intercept of the curves (bottom right), calculated on-the-fly, compared to the retrospectively calculated curves.

FIG. 7 presents plots of values blended according to Eq. (2) that were closer to the reference when negative SURE values were set to zero or to their magnitude for the sanmiguel scene.

FIGS. 8(a)-(d) show a visual representation of MSE for the cropped version of the sanmiguel scene of FIGS. 6 a)-(d), wherein FIG. 8(a) shows the MSE between the denoised image and the reference image, FIG. 8(b) shows the MSE between the reference image and a SURE of the denoised image at 4 sample counts, FIG. 8(c) is Gaussian blur of (b), and FIG. 8(d) is an OIDN of (b), and the figure further presents a plot of the MSE of the SURE values with respect to the actual error calculated retrospectively (bottom left) and a plot of the MSE between the blended image and the reference using: actual error, unfiltered SURE, and filtered SURE.

FIG. 9 presents plots depicting the retrospective mean MSE for ten test scenes using “easy blending” (EB) and blending based on Eq. (2), in accordance with disclosed embodiments, contrasted with the Monte Carlo (MC) estimate and the denoised counterpart: OIDN (left) and RT (right).

FIG. 10 presents plots of MSE results obtained by calculating the convergence curves on the fly using the last two points without filtering error estimates (“Ours (No Filter)”), applying Gaussian blur (“Ours (GB)”), and with OIDN (“Ours (OIND),” the curves labeled as “Ours (GB), den. until 128 spp” represent a scenario in which denoising stops at the sample count of 128, with error estimates filtered using Gaussian blur.

FIG. 11 shows plots of a spectral analysis of the crown scene based on disclosed methods, with the left plot being at sample count 256 and the right plot being at sample count 32k.

FIG. 12 presents plots MSE of methods in accordance with disclosed embodiments, with applied Gaussian blur for filtering error estimates (“Ours”), compared to progressive denoising.

FIGS. 13(a)-(d) present visual comparisons of the Monte Carlo estimates for a cropped version of the image of the bistro-cafe (see FIG. 3), wherein FIG. 13(a) is the image at 256 spp, FIG. 13(b) is an image of results of an OIDN denoiser, FIG. 13(c) is an image denoised by methods in accordance with disclosed embodiments, with Gaussian blur applied for error estimates filtering, and FIG. 13(d) is a 32k spp reference image.

FIGS. 14(a)-(d) present visual comparisons of the Monte Carlo estimates for a cropped version of the image of the crown (see FIG. 3), wherein FIG. 14(a) is the image at 256 spp, FIG. 14(b) is an image of results of an OIDN denoiser, FIG. 14(c) is an image denoised by methods in accordance with disclosed embodiments, with Gaussian blur applied for error estimates filtering, and FIG. 14(d) is a 32k spp reference image.

FIGS. 15(a)-(d) present visual comparisons of the Monte Carlo estimates for a cropped version of the sanmiguel image (see FIG. 3), wherein FIG. 15(a) is the image at 256 spp, FIG. 15(b) is an image of results of an RT denoiser, FIG. 15(c) is an image denoised by methods in accordance with disclosed embodiments, with Gaussian blur applied for error estimates filtering, and FIG. 15(d) is a 32k spp reference image.

FIGS. 16(a)-(e) present visual comparisons of a Monte Carlo estimate for the bathroom2 image of FIG. 3, wherein FIG. 16(a) presents a 1k spp input crop of the image, FIG. 16(b) presents the result of OIDN denoising, FIG. 16(c) presents the result of progressive denoising, FIG. 16(d) presents results generated in accordance with disclosed methods, and FIG. 16(e) shows a 32k spp reference image, and wherein the images corresponding to FIGS. 16(a)-(d) which are identified as “difference” are visual representations of MSE relative to the reference image, and wherein a plot is presented of MSE for the entire image (as opposed to the cropped image).

FIGS. 17(a)-(e) present visual comparisons of a Monte Carlo estimate for the bistro-cafe image of FIG. 3, wherein FIG. 17(a) presents a 512 spp input crop of the image, FIG. 17(b) presents the result of RT denoising, FIG. 17(c) presents the result of progressive denoising, FIG. 17(d) presents results generated in accordance with disclosed methods, and FIG. 17(e) shows a 65k spp reference image, and wherein the images corresponding to FIGS. 17(a)-(d) which are identified as “difference” are visual representations of MSE relative to the reference image, and wherein a plot is presented of MSE for the entire image (as opposed to the cropped image).

FIGS. 18(a)-(b) present a visual comparison of results for the glass of water image of FIG. 3, wherein FIG. 18(a) shows results produced in accordance with disclosed embodiments, applying Gaussian blur to the error estimates (“Ours(GB)”), and FIG. 18(b) shows results produced in accordance with disclosed embodiments, applying OIDN the error estimates (“Ours(OIDN)”), wherein a plot is presented of MSE of these results compared to OIDN denoising.

FIG. 19 shows plots of FLIP scores for the images denoised with OIDN, produced by disclosed methods with Gaussian blur (“Ours GB”) and OIDN (“Ours OIDN”) on estimates, and by progressive denoising (PD). The scores are averaged across all ten Mitsuba scenes, with the left plot being all sample counts and the right plot being higher sample counts.

FIGS. 20 (a)-(f) present visual comparison of denoising methods using input images which are 20×20 crops of the exact center of Mitsuba scenes in FIG. 3, wherein FIG. 20(a) shows the input images rendered at randomly assigned sample per pixel (spp) values, FIG. 20(b) shows results of OIDN, FIG. 20(c) shows results produced in accordance with disclosed embodiments, FIG. 20(d), shows reference images rendered at 32k or 64k spp, FIG. 20(e) shows difference images for OIDN versus the reference images, and FIG. 20(f) shows difference images for results produced in accordance with disclosed embodiments versus the reference images.

FIG. 21 shows visual comparisons of the MC estimate inputs obtained at 512 spp against results generated by OIDN, and disclosed methods relative to the reference at 32K spp, In the disclosed methods, Gaussian blur was applied for error estimates filtering (scenes were generated with AR²T Framework).

FIG. 22 presents plots comparing denoising results produced in accordance with disclosed embodiments (with Gaussian blur applied for error estimates filtering) to various denoising techniques, including progressive denoising (PD), with Monte Carlo Estimate, OIDN, and RT curves being presented for the reference.

FIGS. 23-26 present additional examples of visual comparisons of the Monte Carlo estimates for a cropped version of the images in FIG. 3, laid out similarly to the examples in FIGS. 13-15.

FIG. 27 illustrates the disclosed methods applied to two scenes generated with the AR²T framework from computed tomography (CT) scans: a cone beam CT of a dog torso (“Rocky”), and a spiral CT of a Manix head, with the trend of improved visual quality (quantitative) with comparable or better MSE and FLIP scores observed in the Mitsuba framework is maintained when applied to the AR²T scenes.

FIG. 28 shows, the FLIP score, scene by scene (all ten Mitsuba scenes), corresponding to the averaged scores presented in FIG. 19, for the images denoised with OIDN, produced by disclosed methods with Gaussian blur (“Ours GB”) and OIDN (“Ours OIDN”) on estimates, and by progressive denoising (PD).

FIG. 29 shows the quantitative results of disclosed methods applied to the OptiX denoiser, which are comparable with those obtained for OIDN and RT.

FIG. 30 is a flowchart of a method for rendering three-dimensional (3D) volumes from scan data acquired by computed tomography.

FIG. 31 is a flowchart of a method for denoising images rendered from scan data acquired by computed tomography.

FIG. 32 depicts an embodiment of an advanced radiological imaging system for use with the disclosed techniques for denoising images rendered from scan data acquired by computed tomography.

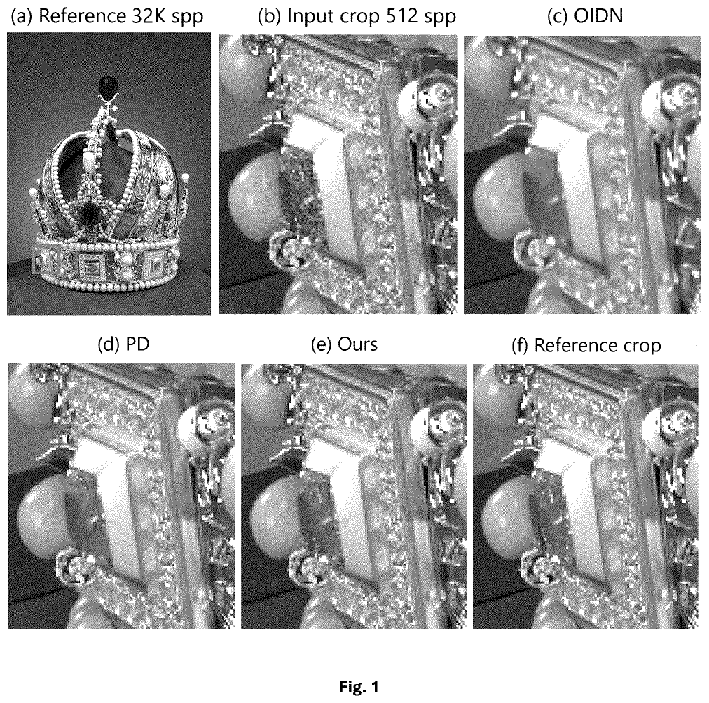

Where considered appropriate, reference numerals may be repeated among the drawings to indicate corresponding or analogous elements. Moreover, some of the blocks depicted in the drawings may be combined into a single function.

### DETAILED DESCRIPTION

Reference will now be made in detail to the embodiments of the invention, examples of which are illustrated in the accompanying drawings. While the invention will be described in conjunction with these embodiments, it will be understood that they are not intended to limit the invention to these embodiments. On the contrary, the invention is intended to cover alternatives, modifications, and equivalents that may be included within the scope of the invention as defined by the appended claims. Furthermore, in the following detailed description, numerous specific details are set forth in order to provide a thorough understanding of the present invention. However, it will be understood by those of ordinary skill in the art that the present invention may be practiced without these specific details. In other instances, well-known methods, procedures, components, and circuits have not been described in detail so as not to unnecessarily obscure aspects of the present invention.

Some of the disclosed embodiments relate to image rendering, e.g., three-dimensional (3D) rendering, of volumes acquired by means of computed tomography (CT), which can be used to illustrate a diagnosis to a patient, train inexperienced clinicians, and/or facilitate surgery planning. The most realistic visualization can be achieved by the Monte Carlo path tracing (MCPT) rendering technique, which is based on the physical transport of light. The inherently stochastic nature of Monte Carlo simulations inevitably introduces noise, particularly in initial iterations and areas with insufficient lighting. Denoising algorithms may be used to reduce noise in the rendered images.

Disclosed embodiments allow for integrating denoising directly into progressive rendering by blending denoised outputs with noisy inputs to enhance visual quality. We introduce a method that predicts the blending weight of denoised images directly during progressive rendering. Our technique adjusts the blending weight for each pixel based on error estimates, effectively “skipping” certain iterations of Monte Carlo Path Tracing (MCPT) and mimicking adaptive sampling a posteriori.

In disclosed embodiments, the objective is to find the weights for unbiased (e.g., noisy) and biased (e.g., denoised) pixels at every Monte Carlo (MC) iteration, which is as close as possible to the reference pixel value. Therefore, we will solve the following optimization problem:

$${arg min}_{{W}_{i} \geq 0} ‖ {r}_{i} ( N ) - \frac{{In}_{i} ( I ) + {W}_{i} {d}_{i} ( I )}{I + {W}_{i}} ‖ \qquad ( 1 )$$

Here, rᵢ(N) denotes the i-th pixel of the reference obtained at iteration N; nᵢ(I) is the i-th pixel of the noisy MC estimate obtained at iteration I, which also represents the weight of nᵢ; Wᵢ denotes the unknown weight of the i-th pixel dᵢ(I) of the denoised image, obtained by applying a denoising algorithm to nᵢ(I), i.e., dᵢ(I)=d (nᵢ(I)). It should be noted that dᵢ(I) is the result of denoising the MC estimate nᵢ(I), as opposed to the result of blending obtained at iteration (I−1). For brevity, we use the term “pixel,” but intend this to mean “pixel radiance.” Furthermore, the reasoning presented below applies to every channel of the pixel: red, green, and blue. Consequently, the blending result of biased and unbiased pixels can be expressed as follows:

$${r}_{i} ( N ) = \frac{{In}_{i} ( I ) + {W}_{i} {d}_{i} ( I )}{I + {W}_{i}} \qquad ( 2 )$$

Another way of determining, for the Monte Carlo simulation, an unbiased MC estimate is given by:

$${r}_{i} ( N ) = \frac{{\sum }_{J = 1}^{N} {n}_{i} ( J )}{N} \qquad ( 3 )$$

Notably, Eq. 2 is essentially a biased MC estimate of pixel i at iteration N=I+Wᵢ. This estimate approaches an unbiased MC estimate as the biased pixel dᵢ(I) approaches the unbiased pixel nᵢ(Wᵢ), obtained at iteration Wᵢ. Below we will first discuss the outcome of “ideal” blending, where the reference image is known, and the problem defined by Eq. 1 can be easily solved. Next, we will analyze the approach using the convergence curves for the practical solution. Finally, we will describe how to compute these curves in real-time alongside the approach for estimating the denoised image.

### Ideal Blending

Analyzing images retrospectively with access to the reference image allows for the calculation of ideal blending between biased and unbiased images. Let RMSEnᵢ and RMSEdᵢ denote the root mean square error (RMSE) values of the MC estimate and denoised pixels, respectively, compared to the reference image. Resolving Eq. 2 for Wᵢ, we get:

$${W}_{i} = I \cdot \frac{{r}_{i} ( N ) - {n}_{i} ( I )}{{d}_{i} ( I ) - {r}_{i} ( N )} = \pm I \cdot \frac{{RMSE}_{{n}_{i}}}{{RMSE}_{{d}_{i}}} \qquad ( 4 )$$

Upon first inspection, one may decide to always choose the plus sign, given that I and RMSE values are positive, and Wi should be positive by the problem definition. However, this solution might not be optimal, given that the RMSE considers only the distance to the reference, not the direction. Therefore, determining the sign in Eq. 4 poses a challenge, as does evaluating the RMSE values. While this formula does not require values of the reference pixel directly, the ambiguity in choosing the sign makes this theoretical solution impractical.

### Convergence Curves

To find a practical solution to the problem posed by Eq. 1, we consider the following optimization problem:

$${min}_{{W}_{i} \geq 0} ‖ {d}_{i} ( I ) - {n}_{i} ( {W}_{i} ) ‖ \qquad ( 5 )$$

Indeed, according to the reasoning behind Eq. 2, where the biased estimate at sample count of N approaches an unbiased MC estimate at sample count of N as long as the biased value at sample count of/approaches the unbiased value at sample count of Wᵢ, the problems defined by Eq. 1 and Eq. 5 are equivalent. It follows from the fact that if we find Wᵢ such that the norm of the difference of dᵢ(I) and nᵢ(Wᵢ) is minimized, then this minimized norm value will directly lead to minimizing the norm value in Eq. 1. Furthermore, for Wᵢ that solves Eq. 5, the following is true:

$$‖ {d}_{i} ( I ) - {n}_{i} ( {W}_{i} ) - {r}_{i} ( N ) + {r}_{i} ( N ) ‖ = 0 ⟹ ‖ ( {d}_{i} ( I ) - {r}_{i} ( N ) ) - ( {n}_{i} ( {W}_{i} ) - {r}_{i} ( N ) ) ‖ = 0 ⟹ ‖ {r}_{i} ( N ) - {d}_{i} ( I ) ‖ = ‖ {r}_{i} ( N ) - {n}_{i} ( {W}_{i} ) ‖ ⟹ {MSE}_{{d}_{i}} = {MSE}_{{n}_{i}} \qquad ( 6 )$$

Here, MSEdᵢ and MSEnᵢ are the mean square errors of denoised pixel dᵢ(I) and MC estimate nᵢ(Wᵢ), respectively, compared to the reference pixel.

In the disclosed approaches, two things should be noted. First, the mean square error of MC estimate compared to the reference can be estimated by its mean variance. Second, the mean variance of pixel values decreases as the iteration number increases, eventually converging to zero. Consequently, each mean variance value corresponds to a specific iteration. With knowledge of the convergence curve, we can determine the iteration at which a particular mean variance value was achieved. Therefore, if we have the mean variance of the denoised pixel, we can assign it the weight it merits in Eq. 2.

It is noted that this practical solution introduces a bias, because while Eq. 5 follows from Eq. 5, the reverse is not necessarily true. However, we will demonstrate that despite this relaxation, the disclosed methods do indeed perform well in practice. In a sense, the disclosed approaches can be viewed as adaptive sampling a posteriori. After denoising, pixels with higher variance improve and receive higher weight, as if they were estimated by more samples.

FIG. 3 displays the mean variance curves in logarithmic scale for a number of scenes generated with Mitsuba, which is an open-source research-oriented rendering system that supports a variety of rendering techniques, including Monte Carlo Path Tracing (MCPT). Ten different scenes were used (from left to right, from first row to third row): (1) “bathroom2,” (2) “bistro-cafe,” (3) “coffee,” (4) “crown,” (5) “glass-of-water,” (6) “kitchen,” (7) “landscape,” (8) “living-room-3,” (9) “sanmiguel,” and (10) “spaceship.” These curves offer valuable insights into the convergence behavior of the Monte Carlo rendering process across various scenes, shedding light on the relationship between iteration count and variance reduction. Despite these curves representing the average mean variance among all image pixels, the same reasoning can be applied for mean variance per pixel.

The mean variance curves inform the disclosed approaches to denoising and adaptive sampling. Notably, all curves in the plot can be effectively approximated by lines of the form:

$$log ( {v}_{I} ) = a \cdot log ( I ) + b \qquad ( 7 )$$

Here, ν₁ represents the mean variance of the entire image obtained on the I-th iteration, and a and b are the slope and intercept of the line, respectively. Note that this I is the same as the one used in Eq. 2, as the weight of an unbiased pixel corresponds to the iteration count. Therefore, it is possible to construct the convergence curve by calculating the slope and intercept of Eq. 7.

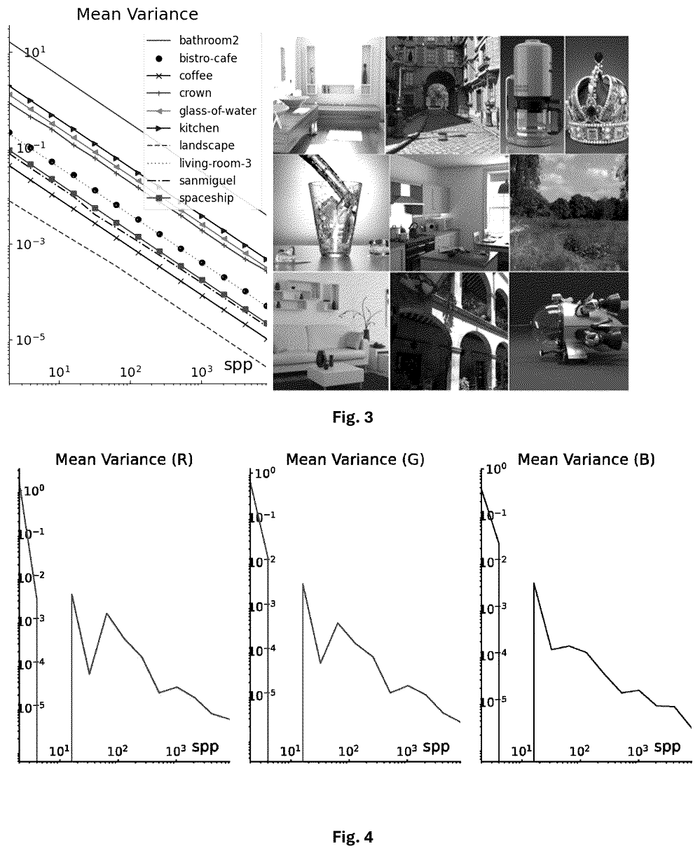

It is important to note that the curves in FIG. 3 represent the average mean variance among all image pixels. However, these curves are much more stable than those of a single pixel, as illustrated in FIG. 4. The jumps at initial sample counts indicate the presence of outliers, such as when a low probability path is found. Therefore, some filtering of the variance might be done before applying the average mean variances approach discussed above.

### Easy Blending

Before describing calculation of the curves, we describe an approach to blending biased and unbiased values involving the following formula:

$${r}_{i} = \{ \begin{matrix}{n}_{i} , & if {RMSE}_{{d}_{i}} > {RMSE}_{{n}_{i}} \\ {d}_{i} , & otherwise\end{matrix} . \qquad ( 8 )$$

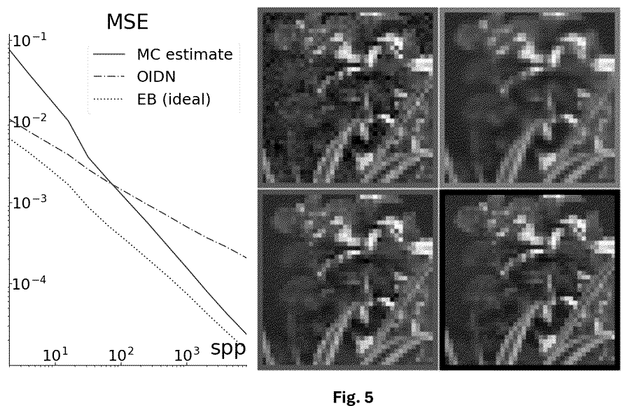

The method, expressed in Eq. 7, does not directly rely on the reference pixel's value. While lacking the precision of Eq. 3, which requires knowledge of exact RMSE values, easy blending offers simplicity in implementation. FIG. 5 presents the mean squared error (MSE) of the MC estimate, its denoised counterpart, and the easy blending solution under the assumption of knowing exact values of RMSEdᵢ and RMSEnᵢ. Based on the findings from FIG. 5, it is noted that both quantitative and visual comparisons for the easy blending solution provide useful results. Consequently, we conducted a practical evaluation, discussed in further detail below, comparing it with the disclosed approaches. Note, that both blending methods discussed above have the advantage of not requiring denoising at every iteration, which allows for the use of denoising performed in earlier iterations.

### Retrospective Curves Calculation

Let's consider a scenario where we have the mean variance values for all rendered images from the initial iterations up to the reference image. In such cases, we can use least squares regression to determine the parameters of Eq. 7. However, due to the lower accuracy of mean variance values in the earlier iterations, some filtering of the data is performed.

Eq. 7 suggests a linear relationship between the points, with the mean variance decreasing as the number of samples increases. Therefore, to achieve the most accurate regression results, we prioritize mean variance values that increase from the last one. This prioritization is based on the statistical principle that the latest value is generally more accurate than previous ones. Additionally, we exclude zero-values from consideration to accommodate logarithm. Following this filtering process, a minimum of two values is sufficient to solve Eq. 7 for the parameters a and b. In embodiments, all pixels, except possibly completely black ones, may have an associated curve calculated retrospectively. In the case of completely black pixels, if denoising algorithms maintain zero-values, an arbitrary weight can be assigned to denoised pixels. However, if zero-values are not maintained, denoised pixels should be assigned a zero weight.

The discussion below considers solutions to the blending problem, such as deciding the blending weights on-the-fly without access to the reference.

### Curves Calculation on the Fly

In practice, we typically only have variance values for previous iterations rather than for all iterations until the reference. Despite this limitation, we can still calculate curves using the available data, although with reduced accuracy. Generally, curves calculated from earlier iterations are less accurate, with accuracy improving as the iterations progress. Operating with this reduced data, it is possible that some pixels may not have enough information to calculate the curve at the current iteration. In such cases, we accept only the last variance value while filtering out all others. Consequently, we need to calculate the second point to enable curve calculation. Let's consider the MC estimate formula:

$$\frac{{\sum }_{i = 1}^{N} {S}_{i}}{N} = R \qquad ( 9 )$$

Here, N represents the number of iterations (samples) Sᵢ for the reference R estimate. We note that:

$$‖ R - {A}_{K} ‖ = ( \frac{N}{K} - 1 ) ‖ R - {A}_{N - K} ‖ , \qquad ( 10 )$$

$$where :$$

$${A}_{K} = \frac{{\sum }_{i = 1}^{K} {S}_{i}}{K} ,$$

$${A}_{N - K} = \frac{{\sum }_{i = K + 1}^{N} {S}_{i}}{N - K} , 1 \leq K \leq N - 1$$

Given that Sᵢ are independent, we have:

$$𝔼 [ {V}_{K} ] = {( \frac{N}{K} - 1 )}^{2} 𝔼 [ {V}_{N - K} ] \qquad ( 11 )$$

Here, V_{K} and V_{N-K} represent the mean variances at iteration K and N−K, respectively. Therefore, if we have the mean variance V_{K} at iteration K, and we set N to be sufficiently large (e.g., 10⁶), we can obtain V_{N-K} and use it for curve calculation without knowing the reference value.

At earliest iterations, the variance of some non-black pixels may still be zero, which means that the paths for this pixel have not picked a light source yet (i.e., because the pixel starts to have some color once the light source is picked). In such a case, the variance would be zero, which would seem to indicate that the pixel has already converged when, in fact, it has not. Therefore, some pre-processing (e.g., filtering) may be used. For example, if the pixels around the pixel in question have already picked the light, then they will have a non-zero variance, so blurring the values will assign some variance also to the non-black pixel with zero variance.

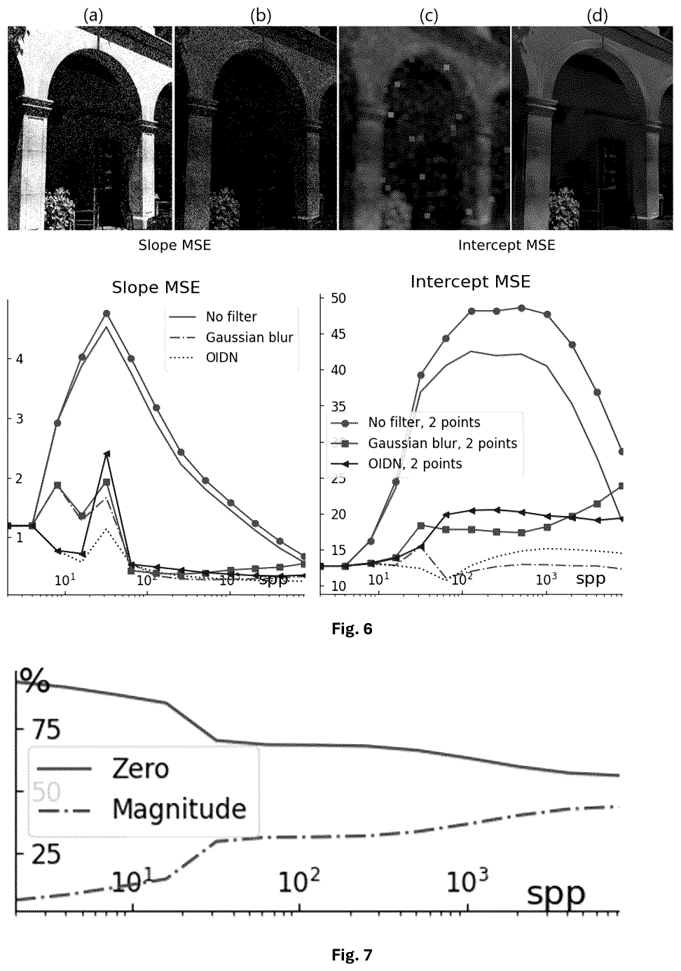

FIG. 6 presents the mean variance of the scene at a sample count of four. To mitigate the noise in mean variance, we apply Gaussian blur. Following, we set σ=min (\sqrt{V̅}, 1)×10², where V̅ is the mean variance of the entire image. It is notable that the mean variance image exhibits high noise, similar to the MC estimate. Therefore, as an alternative to Gaussian blur, we may apply OIDN.

Subsequently, FIG. 6c shows the mean variance filtered by Gaussian blur (window size w=11), and FIG. 6d depicts the mean variance denoised by OIDN. Plots in FIG. 6 demonstrate how denoising improves the MSE of slope and intercept in curve calculation compared to the slope and the intercept of the curves calculated retrospectively. These MSE values show the effectiveness of the denoising methods in preserving the accuracy of the calculated curves. Interestingly, using just two last points for curve calculation does not significantly alter the result. Moreover, the curves appear stable starting from 64 spp.

### Denoising Estimation

We used the Stein Unbiased Risk Estimator (SURE) to assess the quality of the denoised image. For a single denoised pixel i, SURE is expressed as:

$$SURE ( {d}_{i} ( I ) ) ) = - {σ}^{2} + {‖ {d}_{i} ( I ) - {n}_{i} ( I ) ‖}^{2} + 2 {σ}^{2} {d}^{′} ( {n}_{i} ( I ) ) \qquad ( 12 )$$

where nᵢ(I) is distributed normally with variance σ², and the denoised estimator d is weakly differentiable.

While most components of this equation are known, estimating the last term d′(nᵢ(I)) poses a challenge. To address this, we used the Monte Carlo SURE first-order approximation:

$${d}^{′} ( {n}_{i} ( I ) ) \approx \frac{1}{ϵ K} {\sum }_{k = 1}^{K} {b}_{k} ( d ( {n}_{i} ( I ) + ϵ {b}_{k} ) - {d}_{i} ( I ) ) ) \qquad ( 13 )$$

In Eq. 13, bₖ are normally distributed random variables with a mean of zero and covariance equal to 1. Regarding the value of K, we experimentally found that setting it to 1 (for OIDN) or 4 (for RT) is sufficient. A higher count for K indicates lower stability of the denoising algorithm. In experiments, the sensitivity to variance of the OIDN denoiser was found to be twice as high using a CPU compared to using a GPU. We found that setting ∈=1 yields more stable outcomes.

Due to the high variance observed at low sample counts, SURE values may display instability. Additionally, as indicated by Eq. 12, SURE values can potentially be negative, which lacks a physical interpretation since SURE is intended to estimate the mean square distance. During our experiments, we observed that setting negative SURE values to zero improves estimation accuracy at low sample counts. However, this trend shifts at higher sample counts. Additionally, setting SURE to zero at higher sample counts may result in assigning excessive weight to the denoised counterpart, potentially leading to errors if denoising is halted from a certain sample count onwards. Therefore, when the mean SURE value exceeds the mean variance value, we adjust negative SURE values to their magnitude.

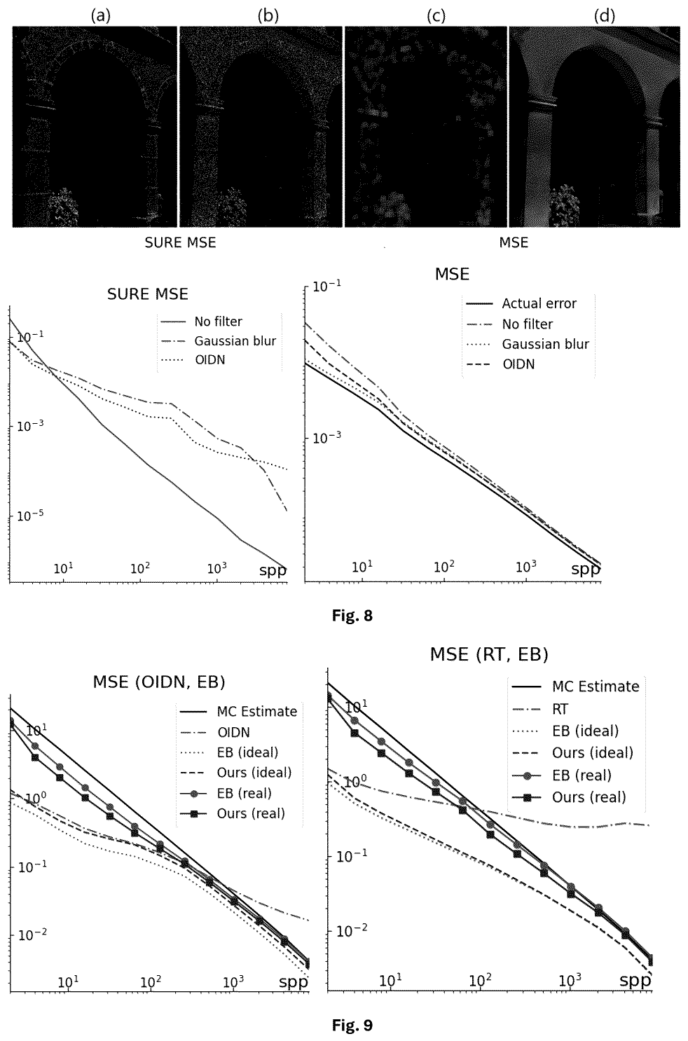

We calculated the weight using SURE set to zero and its magnitude on every MC iteration. Then, we compared the blended value according to Eq. 2 with the reference. The plot of FIG. 7 illustrates the percentage at each sample count of how many blended values were closer to the reference by choosing zero or magnitude for negative SURE. It is noted that setting negative SURE values to zero results in significantly better estimations at lower sample counts, but this tendency decreases as the sample count grows. In some cases, zeroing the negative SURE values produces better results. However, if denoising stalls at particular iterations, the zeroing approach can result in assigning a very high weight to biased pixels, whose quality remains constant as sample counts increase, while the quality of unbiased input improves. Therefore, it is preferable, in some cases, to use the magnitude strategy, which allows unbiased pixels to compete with biased ones. In embodiments, the methods may switch from the zeroing strategy to the magnitude strategy when the mean variance of the MC estimate is less than the mean SURE. As illustrated by FIG. 8, the visual representation of the MSE between the denoised image and the reference appears less noisy compared to the SURE of the denoised image.

To enhance SURE stability, we apply Gaussian blur (w=11, σ=min (\sqrt{V̅}, 1)×10²) as we already do for the mean variance. Additionally, as an alternative approach, we also apply OIDN. The left plot of FIG. 8 illustrates the MSE between SURE values and actual errors, indicating improvement at lower sample counts. However, the right plot shows that the MSE between the blended image and the reference benefits from filtering for a longer period. Observing both FIGS. 6 and 8, we notice a similar behavior in OIDN's performance as observed in the denoising of the MC estimates. While the Gaussian blur effect is constrained by the choice of σ, OIDN can sometimes yield inferior results at higher sample counts compared to unfiltered values. Employing the same principle applied for Gaussian blur, we halt the denoising of mean variance and SURE when the mean variance of the entire image falls below mean SURE.

With these components in place, we calculate the weight of the denoised pixel for blending defined by Eq. 2 using:

$$log ( {W}_{i} ) = \frac{log ( {SURE}_{i} ) - b}{a} , \qquad ( 14 )$$

where a and b define the convergence curve Eq. 7.

The convergence of the blending result directly follows from the method's properties. With increasing sample count, the accuracy of the mean variances of MC estimates improves, leading to more reliable estimates for blending. Furthermore, the distribution of the sample mean approaches a normal distribution, which is a well-suited condition for applying SURE for optimal weighting. Since SURE is an unbiased estimator, it minimizes the risk of the blended image, ultimately leading to convergence of the blending process.

### Results

We present our findings based on ten scenes rendered at sample counts ranging from 2 to 65536 samples per pixel (spp), as depicted in FIG. 3. These scenes are sourced from publicly available pbrt scenes and were rendered in Mitsuba.

All of the examples include analyzing the scenes rendered at 2, 4, 8, 16, 32, 64, 128, 256, 512, 1024, 2048, 4096, and 8192 samples per pixel (i.e., powers of 2 from 2 to 8192), together with their corresponding albedo, normal, and mean variance. We do not include the images rendered at 16384 spp as not all scenes are provided with the rendering at this sample count. The scenes rendered at 32K and 64K spp are used as references. The references themselves are excluded from the analysis, because the reference image would be compared to itself, yielding no difference.

Thus, the rendered scenes were analyzed at different levels of detail, which was achieved by varying the number of samples per pixel (spp). Samples per pixel refers to the number of times the renderer calculates the color of a pixel in a scene. The higher the spp, the more detailed and accurate the image is, but it also requires more computing power and time. The scenes are analyzed at varying levels of detail, from 2 samples per pixel (i.e., very low detail) to 8192 samples per pixel (i.e., very high detail). In addition to the images themselves, their corresponding albedo, normal, and mean variance were also analyzed. Albedo refers to the measure of the diffuse reflection of light from a surface, i.e., it is a measure of how much light that hits a color will become a part of that color. Normals are vectors that are perpendicular to a surface, used to determine the orientation of a surface relative to a light source or viewing perspective.

We evaluated disclosed methods using two denoising algorithms: OIDN with albedo and normal, and RT—a denoiser based on OIDN but operated on albedo, normal, and input mean variances. RT is a generic ray tracing denoising filter based on a convolutional neural network (CNN) which is suitable for denoising images rendered with Monte Carlo ray tracing methods.

FIG. 9 illustrates the mean MSE retrospectively calculated for all test scenes using easy blending and blending according to Eq. 2, compared with the MC estimate and the denoised counterpart. The “ideal” curves correspond to blended images obtained using retrospectively calculated curves and estimating the denoised image with actual MSE with the reference images. The “real” curves represent blended images obtained using mean variances and SURE. Despite the easy blending curves being better than the curves according to Eq. 2 when calculated retrospectively, the situation changes when blended using error estimates, demonstrating that the curves approach outperforms easy blending for real scenarios.

FIG. 10 shows the results achieved by calculating the convergence curves on the fly using only the last two points, with variances both unfiltered and filtered through Gaussian blur, as well as with OIDN. Similarly, SURE values were examined without any filtering, filtered with Gaussian blur, and processed with OIDN. Additionally, the plots depict the scenario where denoising halts at the sample count of 128, with both mean variance and SURE values filtered using Gaussian blur. It is noted that the filtering substantially enhances the results and, even in cases where denoising terminates at a lower sample count, the curve smoothly aligns with the reference alongside the MC estimate.

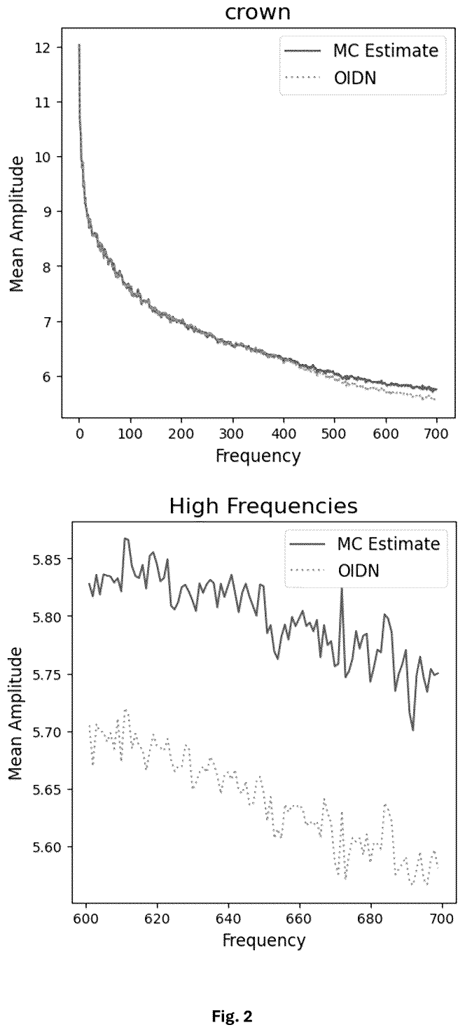

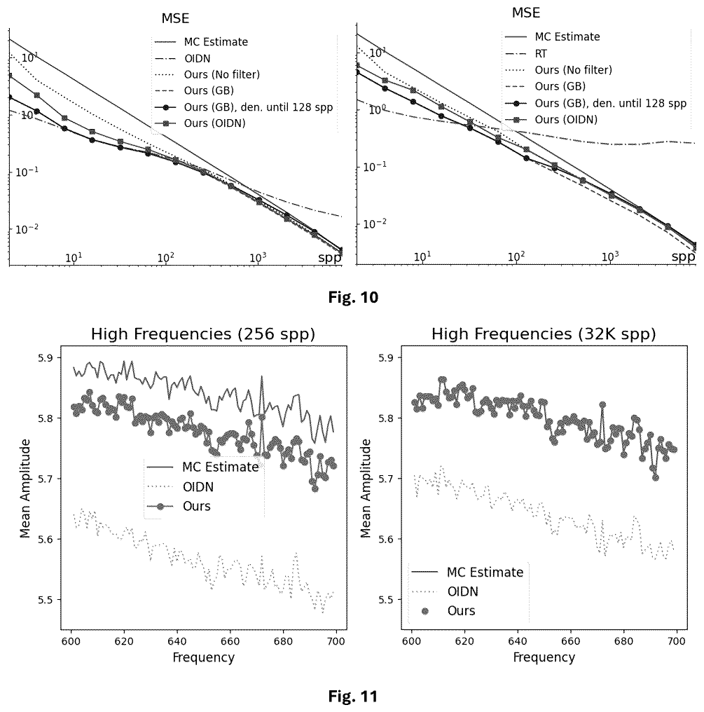

In one experiment, the spectral analysis performed for the Crown scene was repeated, as illustrated in FIG. 2, with the curves calculated on-the-fly and the estimates filtered with Gaussian blur. As FIG. 11 demonstrates, the method successfully reduces noise at the lower sample counts while maintaining the amplitudes of the higher frequencies at the higher sample counts, in contrast to the OIDN, which denoises too aggressively in this example.

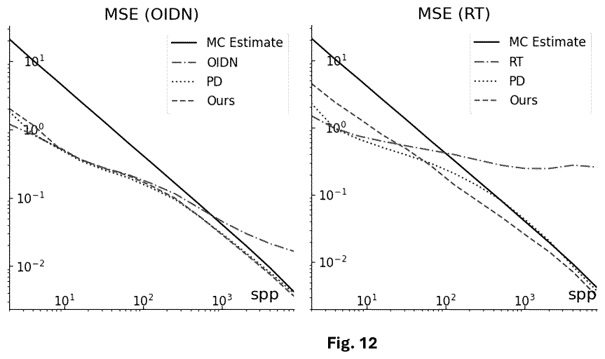

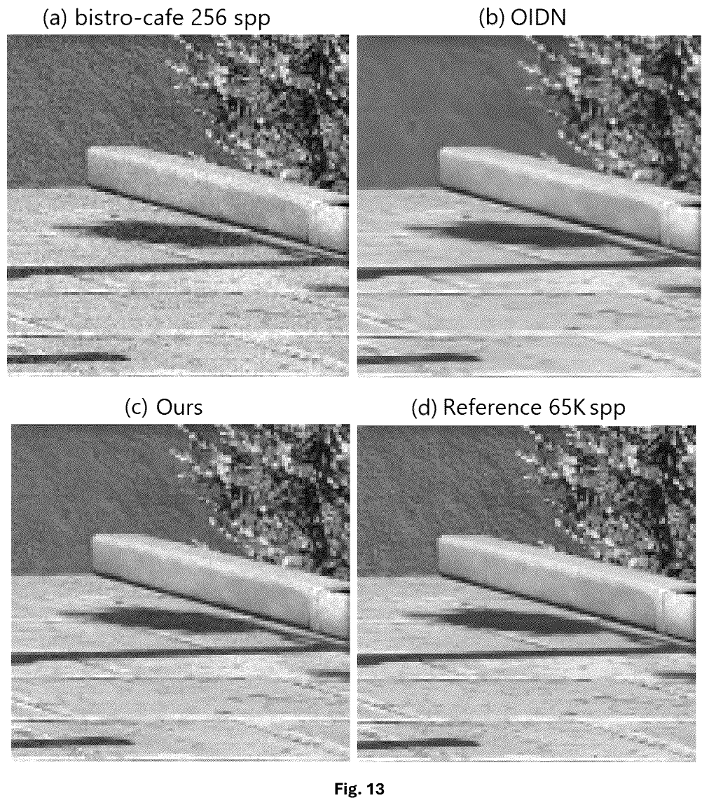

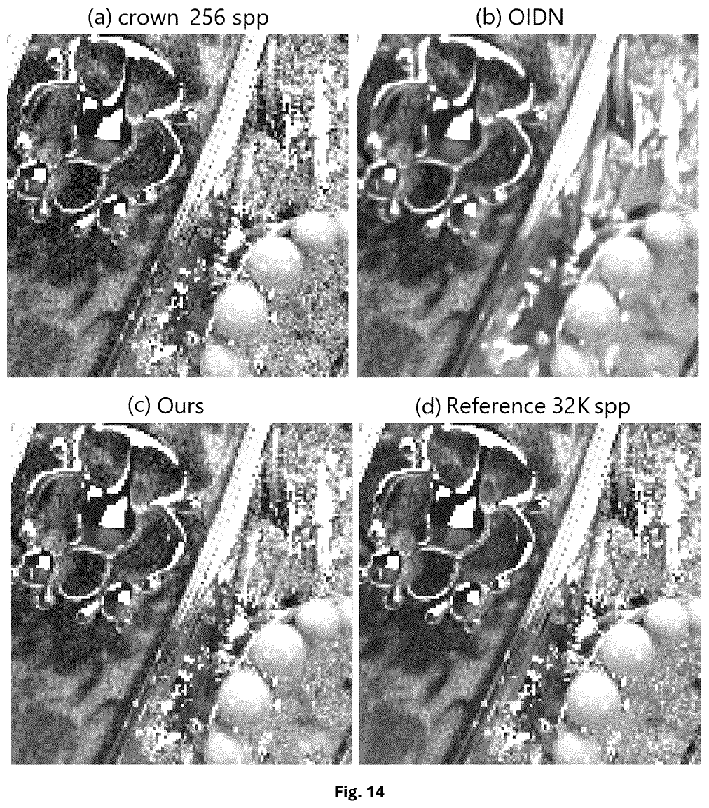

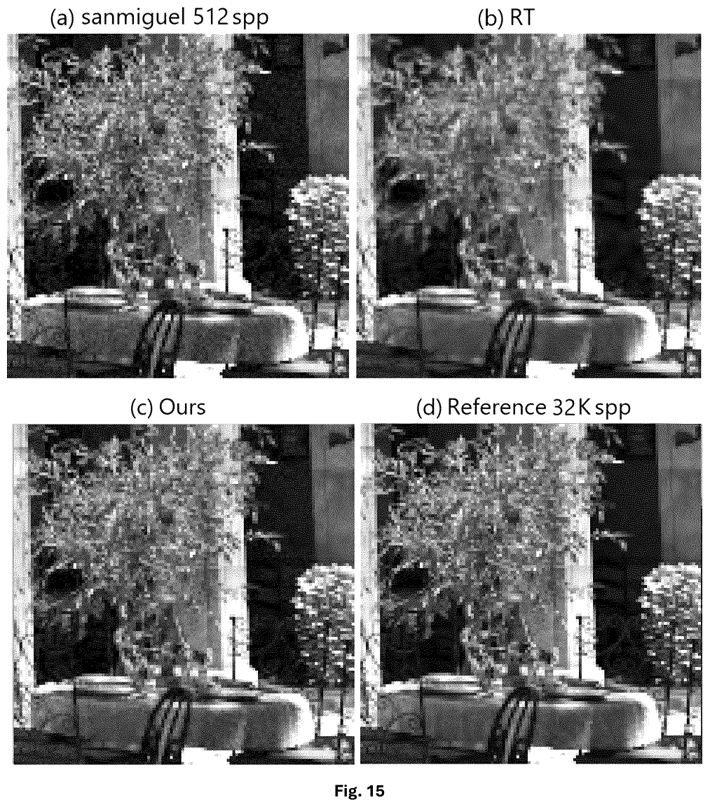

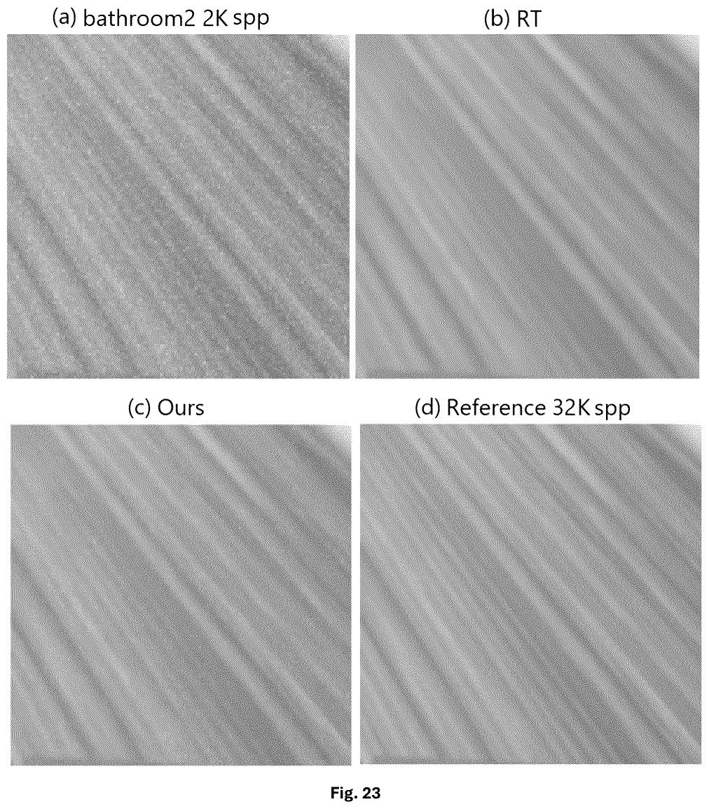

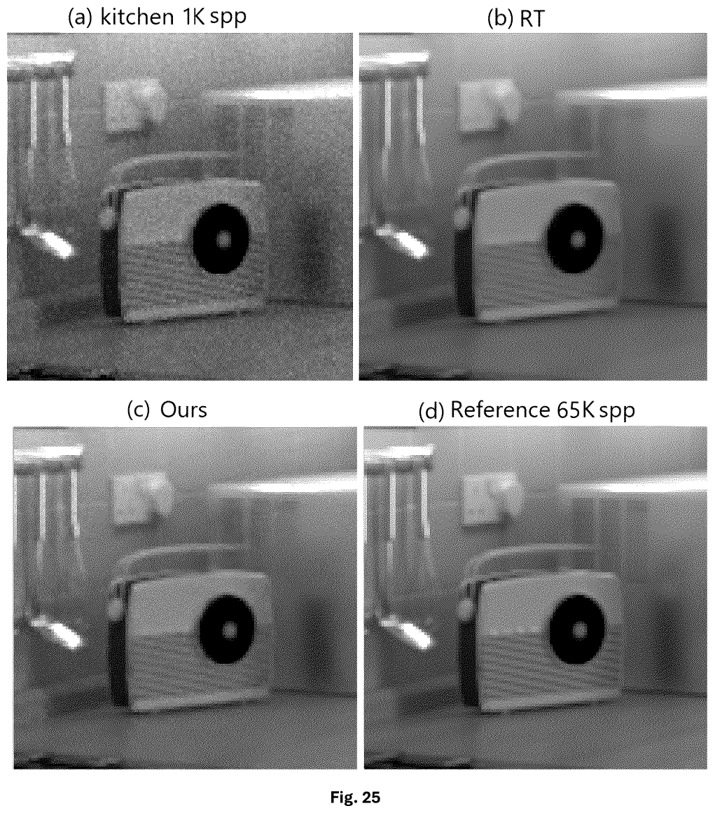

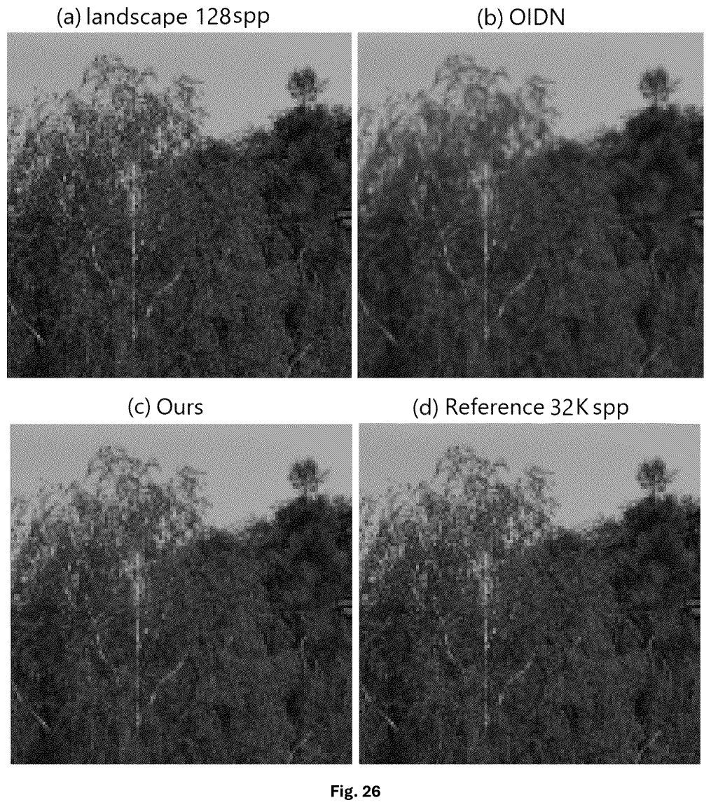

FIGS. 12, 16, 18 provide a comprehensive visual comparison of the effectiveness of disclosed methods in contrast to OIDN and RT denoisers. The visual comparison showcases how the disclosed methods excel in enhancing the level of detail while effectively preserving denoising in areas where it is important. Additional examples are shown in FIGS. 23-26.

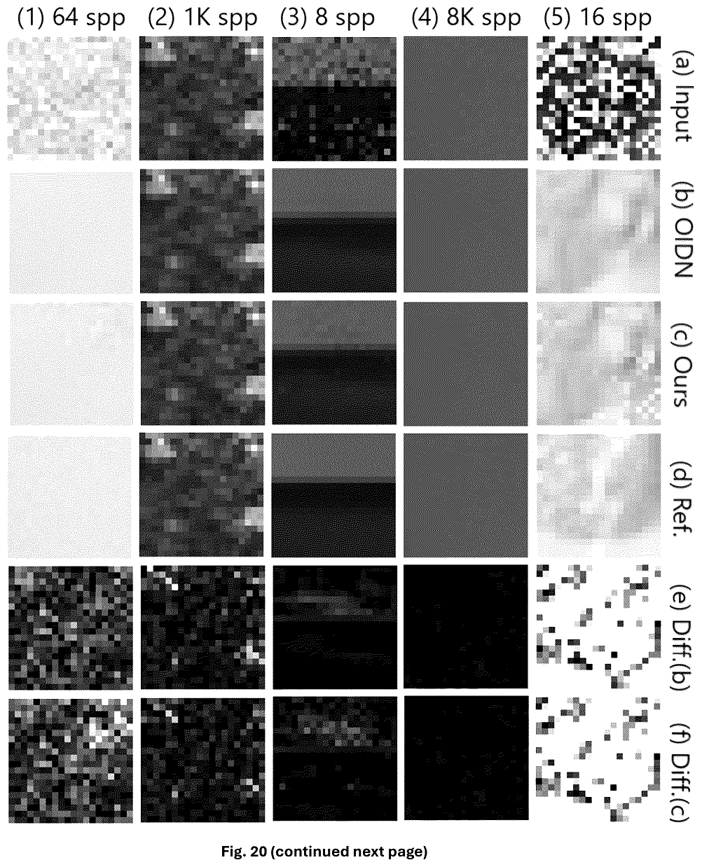

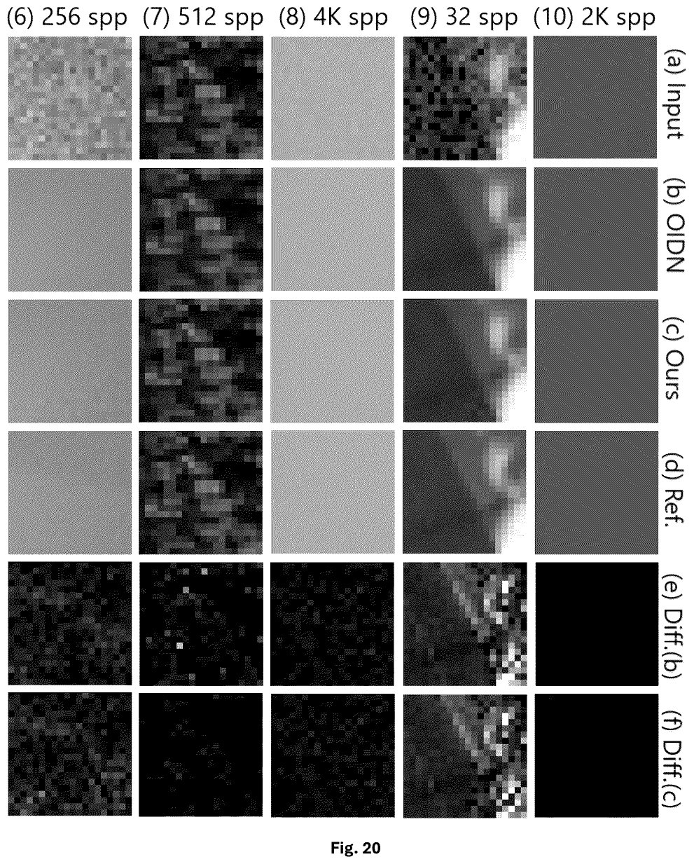

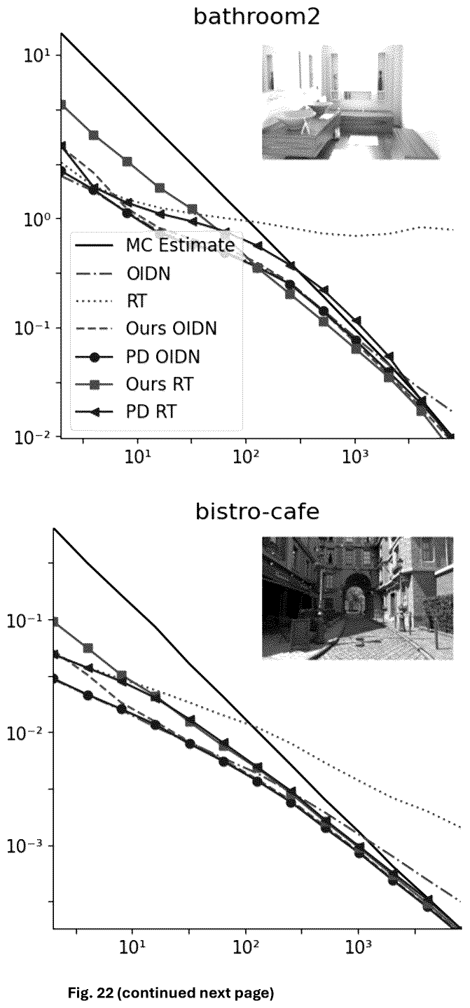

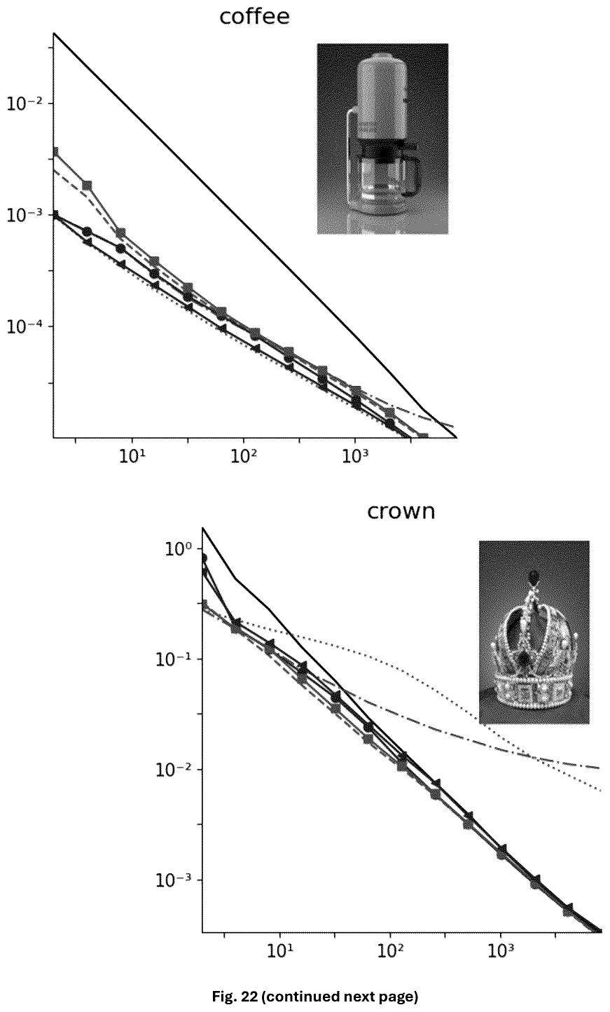

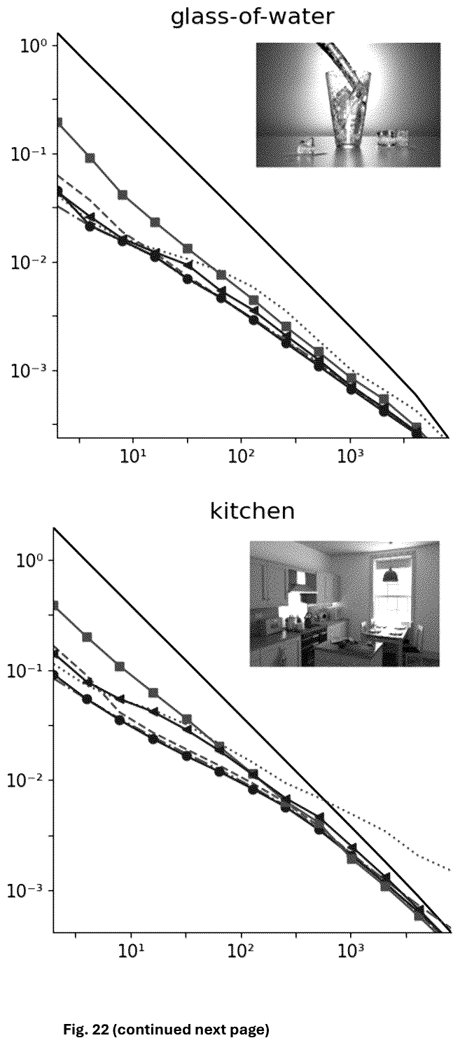

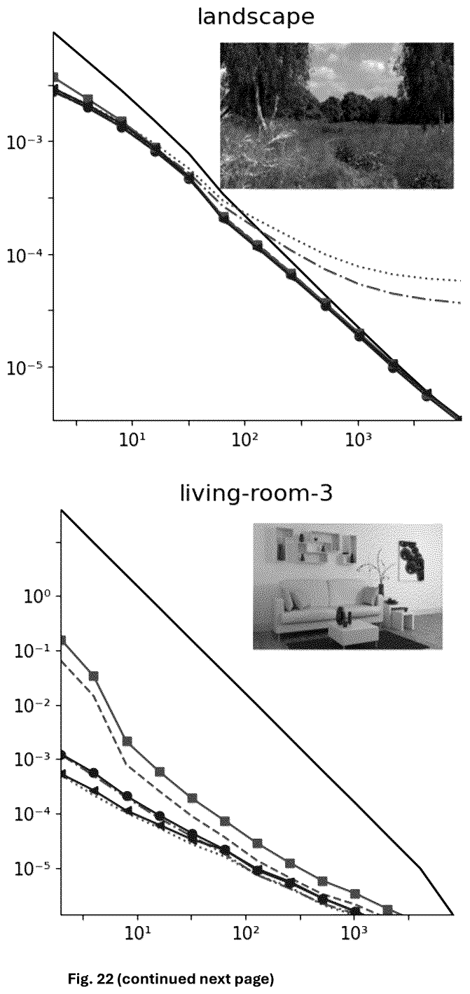

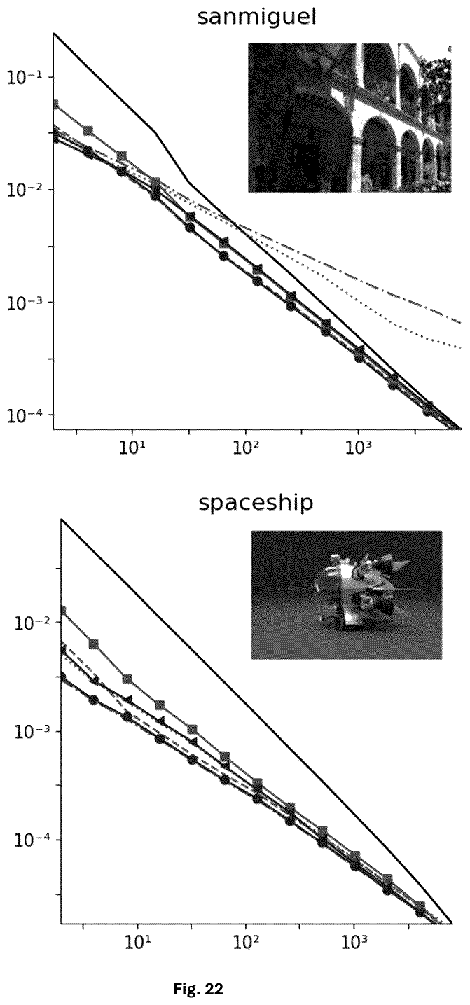

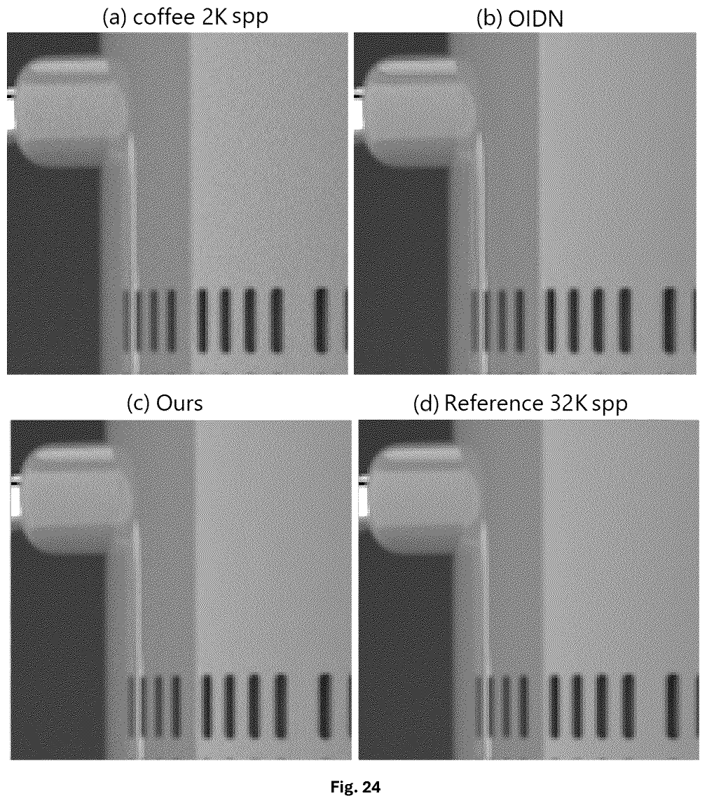

Furthermore, we present a comparative analysis of the results obtained from our method, denoising algorithms, and progressive denoising (PD) technique. FIG. 12 provides an overall quantitative comparison. We observe comparable results when using OIDN for image denoising, while our method outperforms progressive denoising when using RT, particularly starting from 64 samples per pixel. The comparison scene by scene can be found in FIG. 24. FIGS. 20, 22 give a visual comparison of our method with OIDN, RT, and PD. Through a graphical representation of MSE concerning the reference image, the plots illustrate the quantitative differences between the methods.

On our Nvidia RTX A4000 GPU, denoising an image of 1920×1080 using OIDN takes approximately 15 ms, while SURE calculation takes about 16 ms for K=1 for the pre-generated randoms bk used in Eq. 13. Curves calculation using two points and eventual Gaussian blur takes less than 1 ms. In total, producing the blending result takes about 35 ms.

### Discussion and Limitations

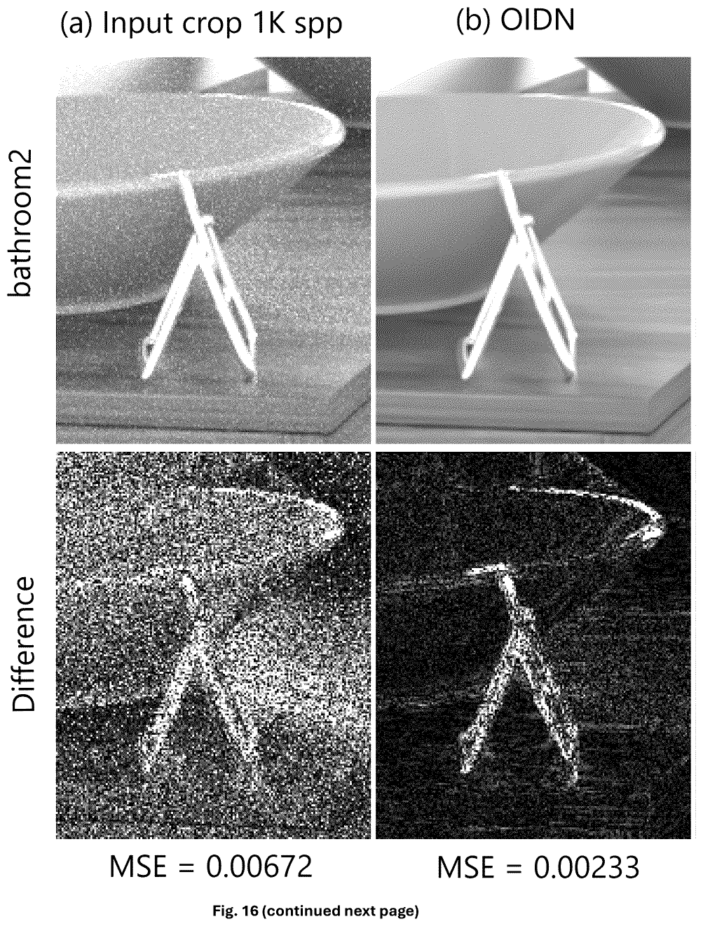

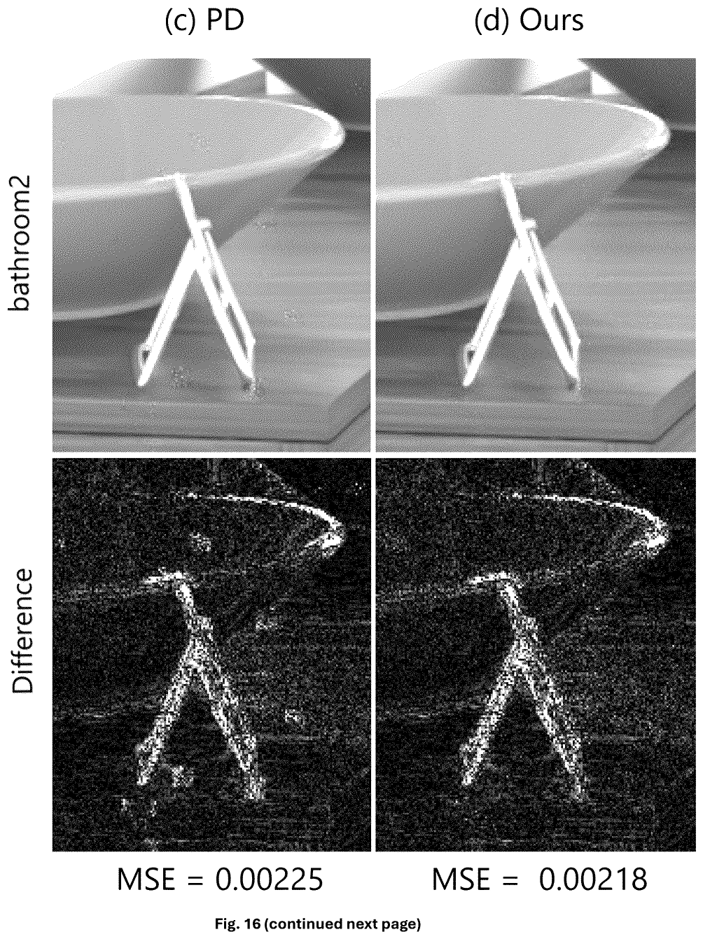

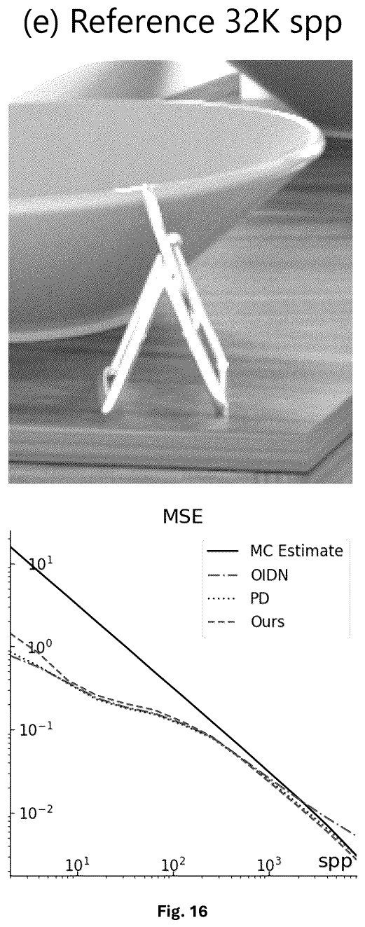

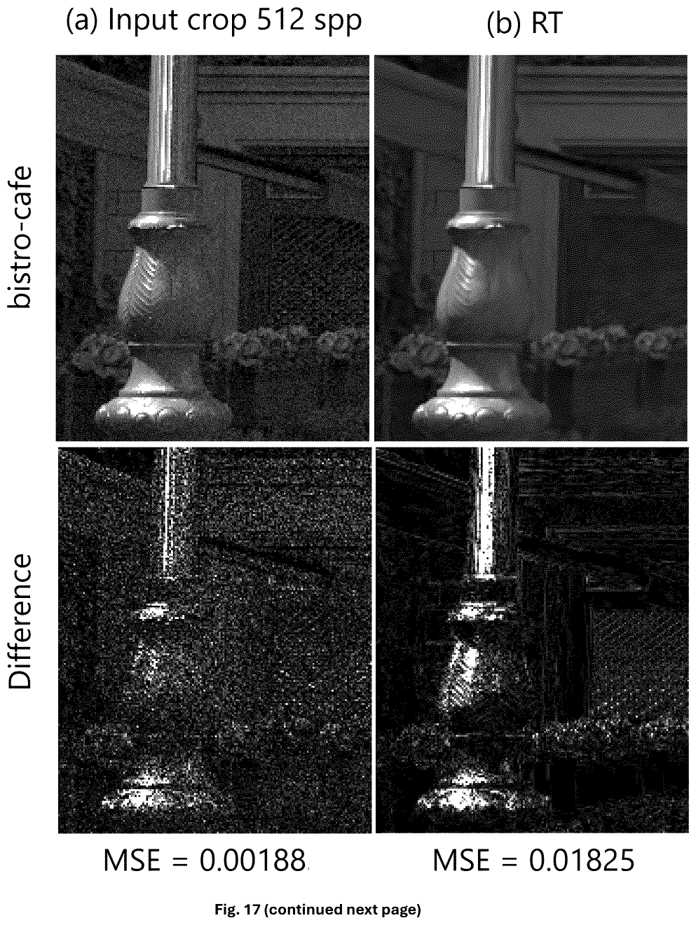

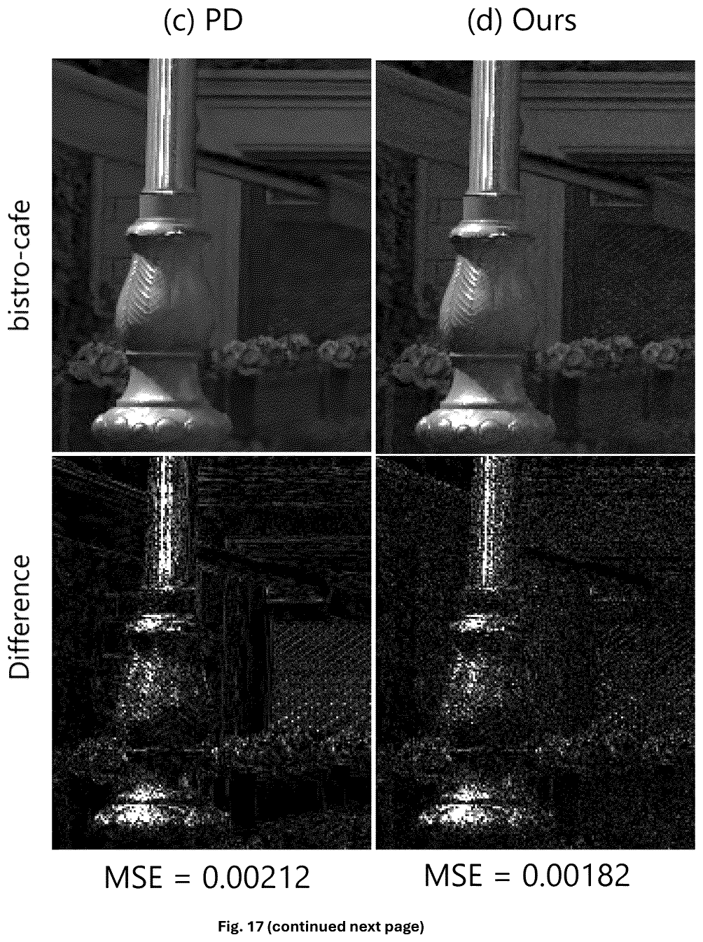

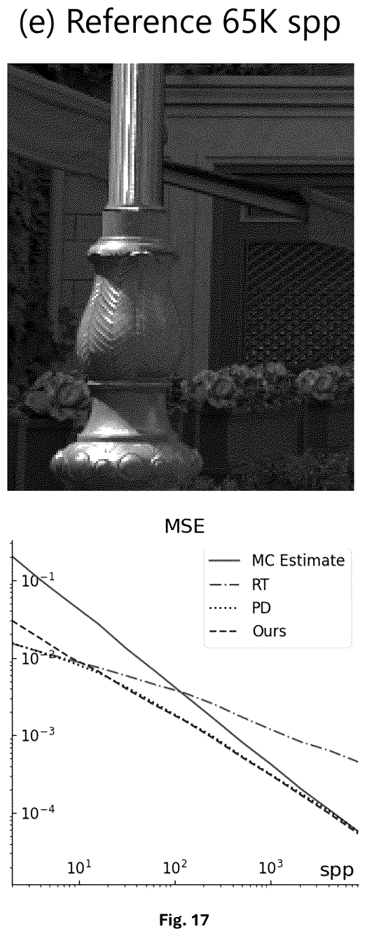

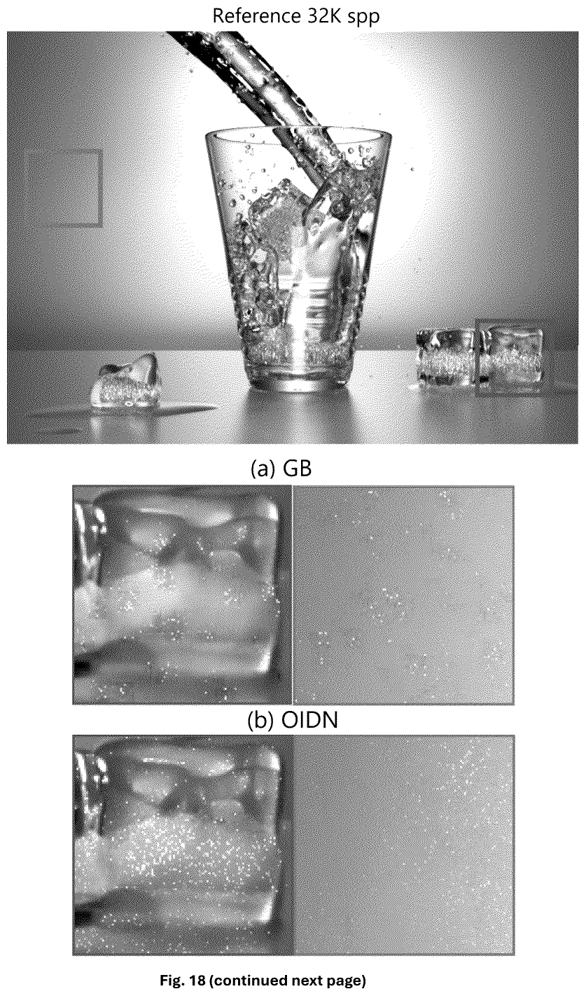

The results show that leveraging denoising of the mean variance and SURE yields quantitative outcomes comparable to, or even superior to, those achieved by the PD network, as evidenced, e.g., in FIGS. 16 and 17. However, our method requires a reasonable number of iterations of MCPT for computation. First, because the input radiance values of MC images should adhere to a normal distribution, to provide for SURE's accuracy, and the accuracy of the estimate increases with the sample count. Second, because of the accuracy of mean variances needed for curves calculation. The disclosed methods can be applied at any iteration, but they start excelling from sample counts 16 in our experiments. Applying Gaussian blur to mean variance and SURE proves advantageous for reducing estimation errors at lower sample counts. Denoising SURE with OIDN yields inferior quantitative results, although visually, the blended images appear more natural and aesthetically pleasing, exhibiting less artificiality, as illustrated by FIG. 18. This result may be explained by the fact that the MSE metric is too basic to capture visual perception. To address this gap, we considered HDR-FLIP, which is an error metric designed to evaluate the perceptual quality of rendered high dynamic range images.

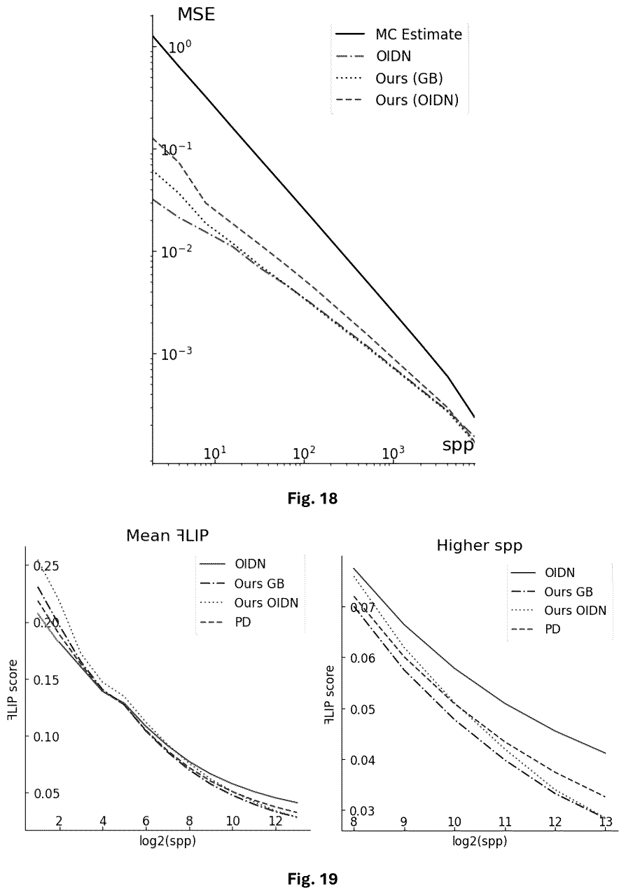

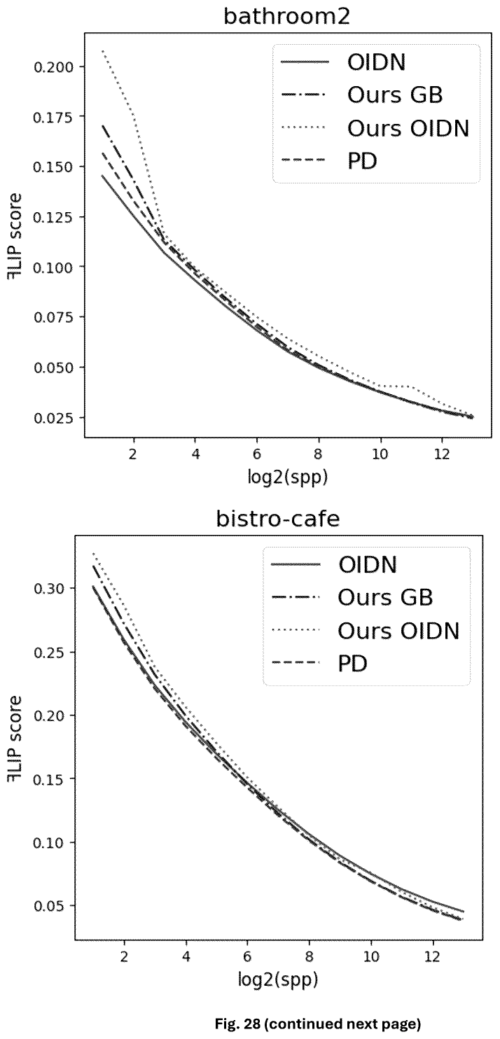

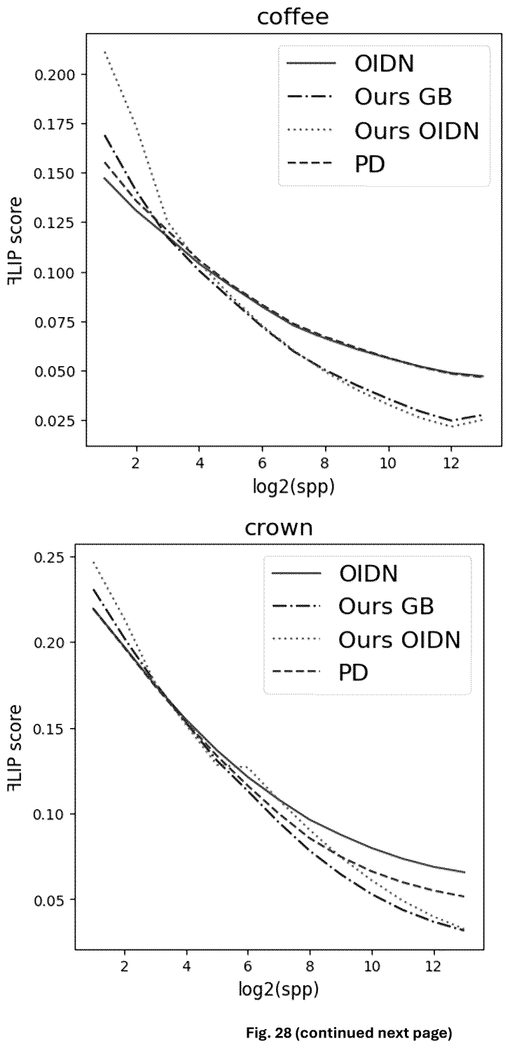

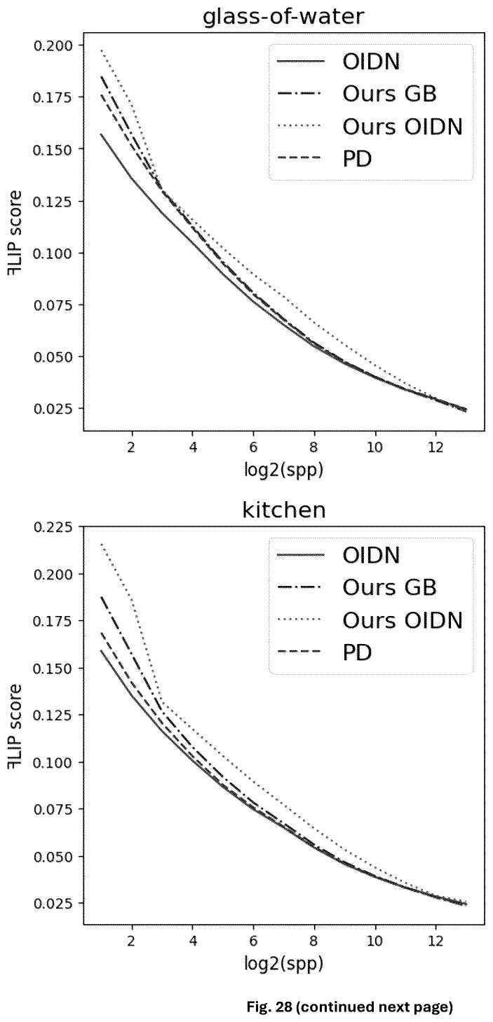

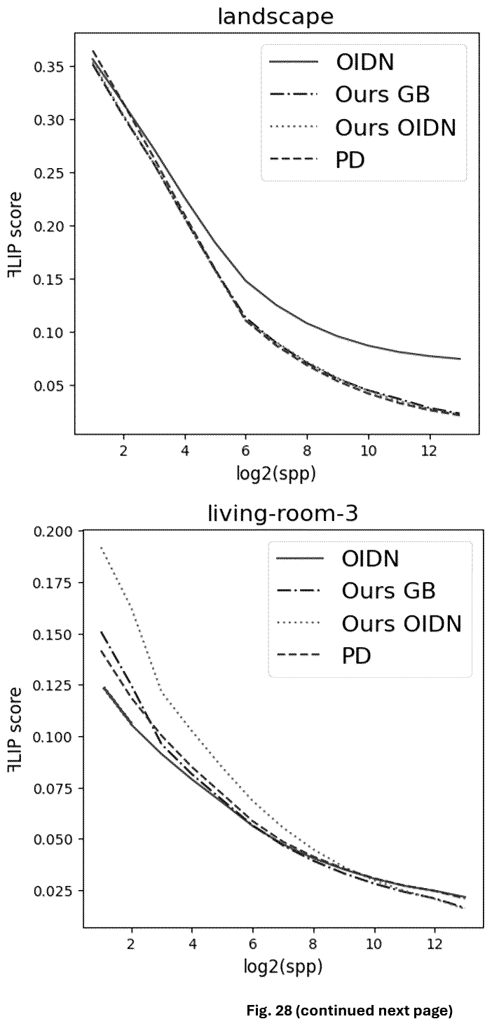

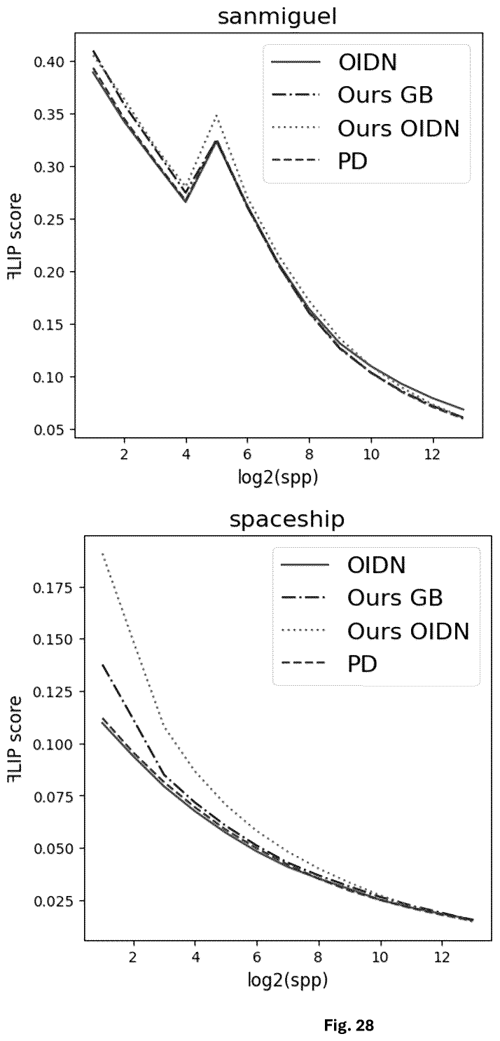

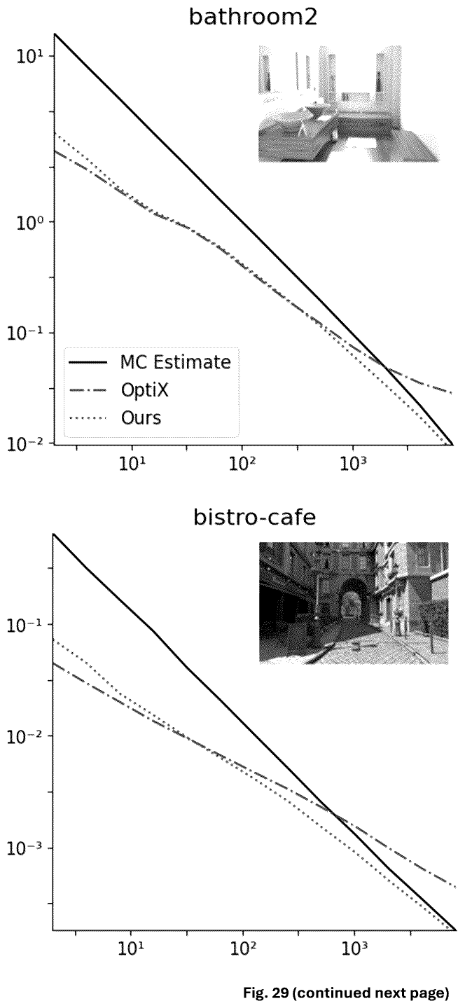

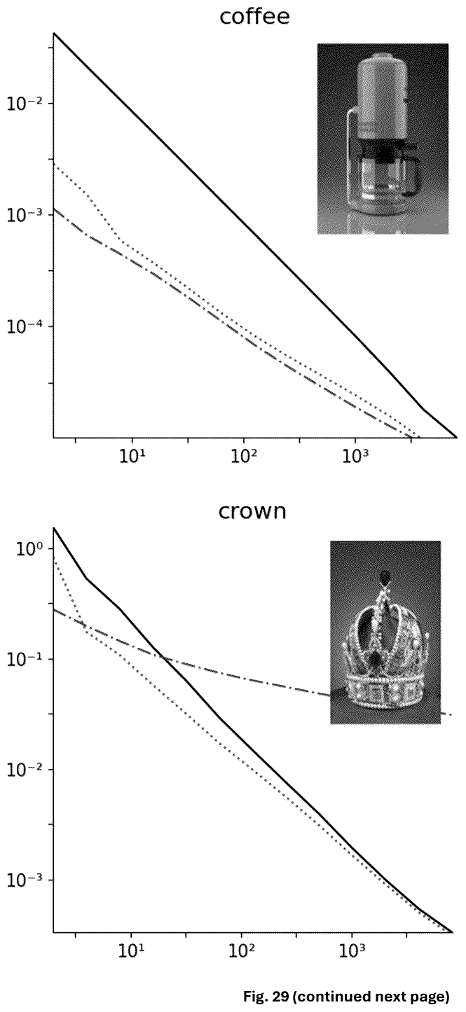

As FIG. 19 shows, the FLIP score, averaged across all ten scenes, is lower for OIDN at lower sample counts (up to spp 16). The curve representing disclosed methods with Gaussian blur on estimates overlaps with that of progressive denoising (PD) (up to spp 128), becoming better at higher sample counts (from spp 256). However, the method using OIDN on estimates performs worse than the one with Gaussian blur, even in terms of average FLIP scores. This discrepancy may be due, in part, to inherent limitations of metrics which evaluate a single image, rather than a temporal sequence produced during progressive rendering. For the FLIP scores, scene by scene, see FIG. 28.

As demonstrated by FIG. 20, disclosed methods, as in deep-learning based approaches, are not devoid of artifacts. These artifacts are more noticeable at low sample counts (i.e., when the image's detail level is low) and on homogeneous areas (i.e., areas with uniform features), as exemplified by scene crops (3) and (9). In highly detailed areas, as exemplified by crops (2) and (7), the disclosed approaches perform particularly well. However, considering the presence of artifacts at lower sample counts and the MSEHLIP score results, some embodiments may start applying blending techniques from a sample count determined empirically. Alternatively, blending may be applied when the mean variance of the MC estimate becomes less than the mean SURE.

The successful on-the-fly calculation of curves is evident, with the primary challenge lying in precise SURE calculation—better precision leading to superior results. In embodiments, denoising-aware adaptive sampling for Monte Carlo ray tracing may be used to enhance outcomes. Furthermore, considering the performance of OIDN in variance and SURE denoising, a dedicated denoising algorithm for the estimators could be used.

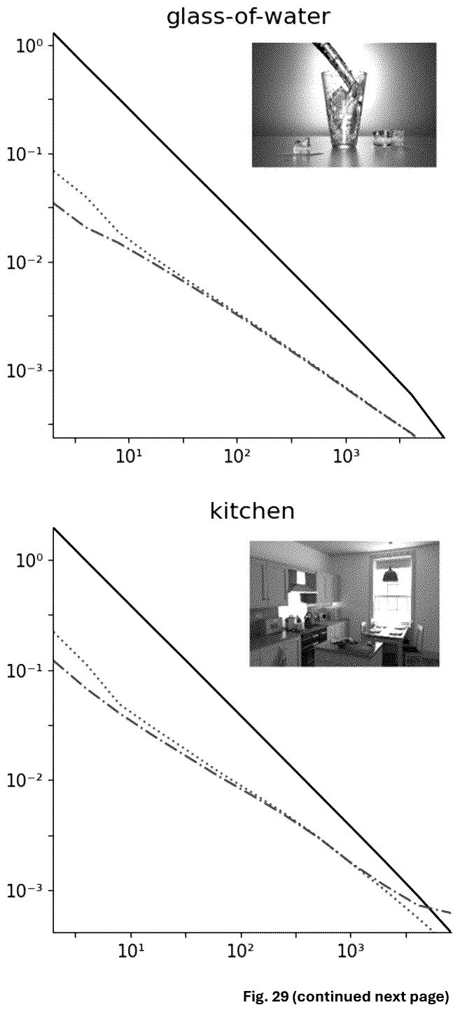

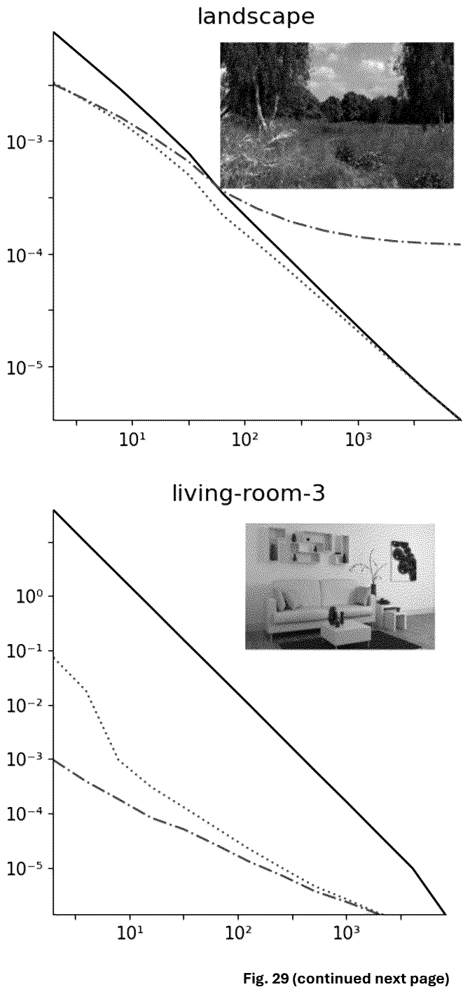

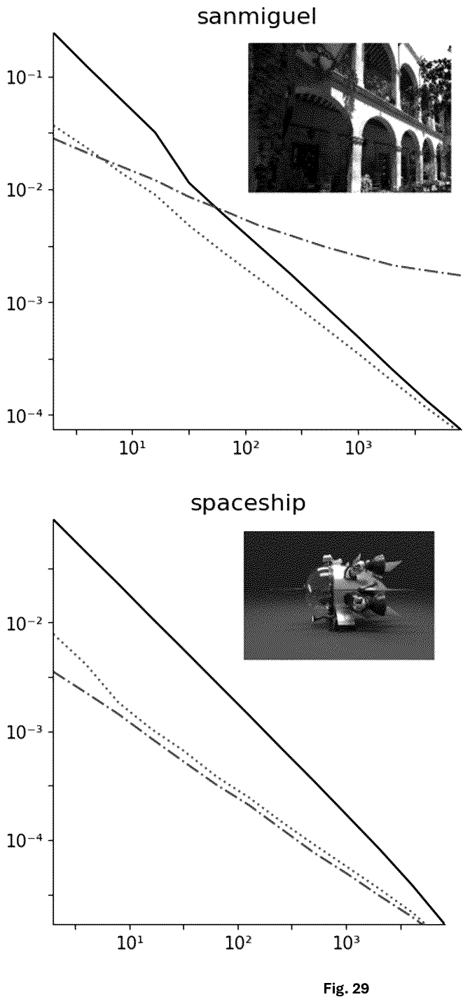

Experimental results were obtained using OIDN and OIDN-derived denoisers. However, disclosed methods are not dependent on the choice of the denoising algorithm, except for the requirement, in some embodiments, that the denoising operator should be weakly differentiable, as imposed by SURE. As shown in FIG. 29, further comparisons may be seen in the quantitative results of disclosed methods applied to the OptiX denoiser. Despite OptiX having different characteristics from OIDN, the results are comparable with those obtained for OIDN and RT. In embodiments, the disclosed methods may be applied to non-deep-learning-based denoisers, such as spatiotemporal variance-guided filtering or its derivatives.

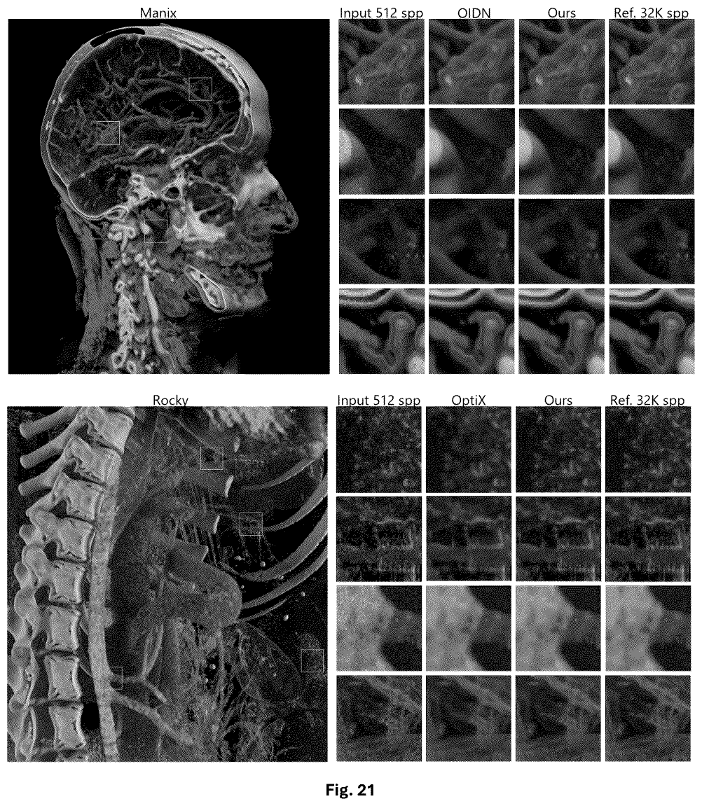

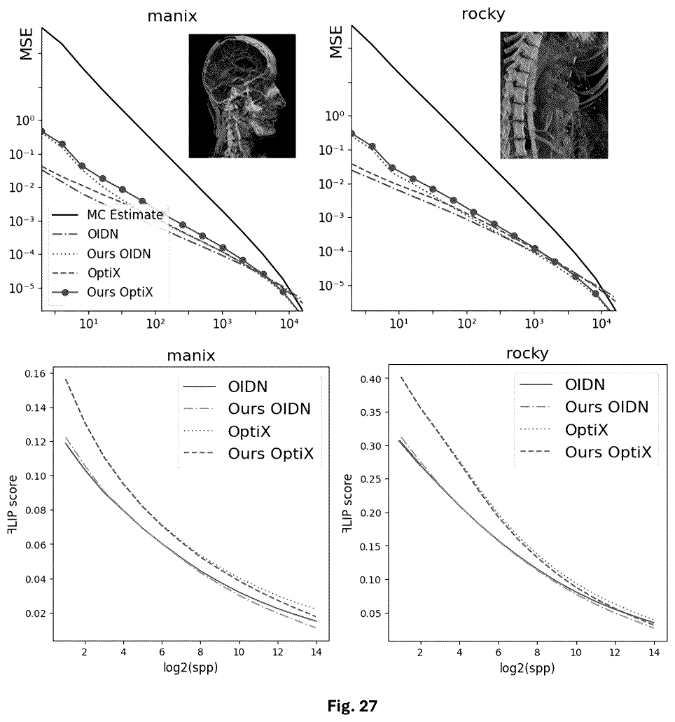

Although we primarily demonstrated the methods on scenes generated with the Mitsuba framework, the methods are not limited to a specific MCPT framework. In fact, some embodiments may only require that the samples follow a normal distribution. To demonstrate this broader applicability, we also successfully applied disclosed methods to two scenes generated with the AR²T framework from computed tomography (CT) scans: a cone beam CT of a dog torso (“Rocky”) available on Kaggle (anonymized), and a spiral CT of a Manix head available at public.sethealth.app/manix.raw.gz. The trend of improved visual quality with comparable or better MSE and FLIP scores observed in the Mitsuba framework is maintained when applied to the AR²T scenes, as shown in FIG. 21 (qualitative) and FIG. 27 (quantitative).

While our results are demonstrated on pre-generated scenes, considerations for real-time MC rendering are important. Firstly, determining the optimal number of mean variance buffers for curve calculation is performed. Although our experiments used mean variances at various sample counts, practical applications may use different steps and intervals for curve updates. Secondly, indicators for denoising cessation, such as the mean variance of the entire image becoming lower than the mean of SURE, could be used.

Additionally, disclosed embodiments may be applied to adaptive sampling techniques, wherein weights may be assigned to each pixel to guide the process. Moreover, in many types of sampling algorithms, in addition to denoising algorithms, it is necessary to determine whether their estimates are sufficient, not only for adaptively selecting new samples, but also for combining two estimates with different amounts of variance using an appropriate weight. For example, in Reservoir-based Spatio-Temporal Importance Resampling (ReSTIR), calculating blending weights based on variance estimation can enhance the process by providing a more robust mechanism for importance resampling. The methods disclosed herein may be applied in these areas, beyond the specific context of denoising.

### Conclusion

Disclosed embodiments provide a method to predict the weight of denoised images during progressive rendering. This process involves “skipping” a specific number of MCPT iterations, determined through denoising. We dynamically adjust these weights for each pixel based on denoised variance, effectively simulating adaptive sampling a posteriori. Among the advantages of the disclosed approaches is that it provides an analytical method which achieves precise convergence of the blended output to the reference image. The disclosed methods, unlike deep-learning techniques, allow for integration with any denoising algorithm. Embodiments described herein illustrate that the disclosed methods can be seamlessly incorporated into pre-trained or analytical denoisers, significantly enhancing visual quality without the need for denoising at every MCPT iteration. This not only streamlines the rendering process, but also enhances efficiency and output fidelity.

### Rendering 3D Scan Data

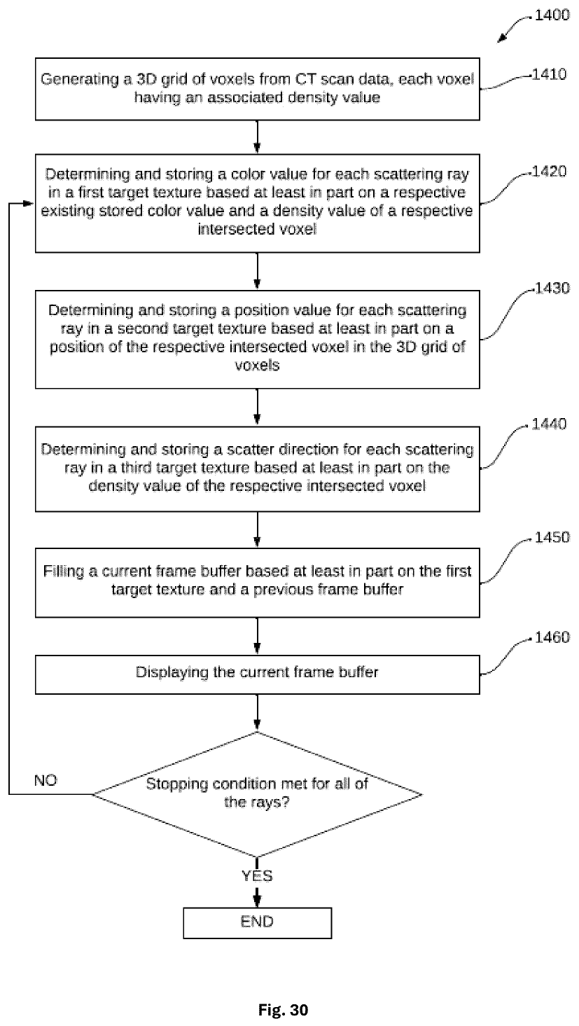

FIG. 30 is a flowchart of a method 1400 for rendering three-dimensional (3D) volumes from scan data acquired by computed tomography (CT). The method includes generating a 3D grid of voxels from CT scan data, each voxel having an associated density value (1410). The method further includes iteratively tracing a plurality of rays originating at a camera position by performing the following. A color value is determined and stored for each scattering ray in a first target texture based at least in part on a respective existing stored color value and a density value of a respective intersected voxel (1420). A position value is determined and stored for each scattering ray in a second target texture based at least in part on a position of the respective intersected voxel in the 3D grid of voxels (1430). A scatter direction is determined and stored for each scattering ray in a third target texture based at least in part on the density value of the respective intersected voxel (1440). A current frame buffer is filled based at least in part on the first target texture and a previous frame buffer (1450). The current frame buffer is displayed (1460). The method further includes repeating (1420-1460) until a stopping condition is met for all of the rays of the plurality of rays (1470).

In embodiments, the method may further include determining an initial origin and direction for each ray of the plurality of rays based at least in part on the camera position. In an iteration, when a scattering ray does not intersect a voxel or a light source, a stopping condition of the scattering ray may be met and a zero-length vector may be stored in the third target texture. When a scattering ray intersects a light source, a stopping condition of the scattering ray may be met and the respective existing stored color value may be attenuated based at least in part on the color of the intersected light source. When a scattering ray exceeds a scatter number limit, a stopping condition of the scattering ray may be met and the respective existing stored color value may be set to zero.

In embodiments, the first target texture, the second target texture, and the third target texture, may each be two-dimensional textures of viewport size. wherein the filling of the current frame buffer based at least in part on the first target texture and the previous frame buffer comprises summing corresponding values of the first target texture and the previous frame buffer and dividing by the number of iterations.

### Denoising Images Rendered from CT Scan Data

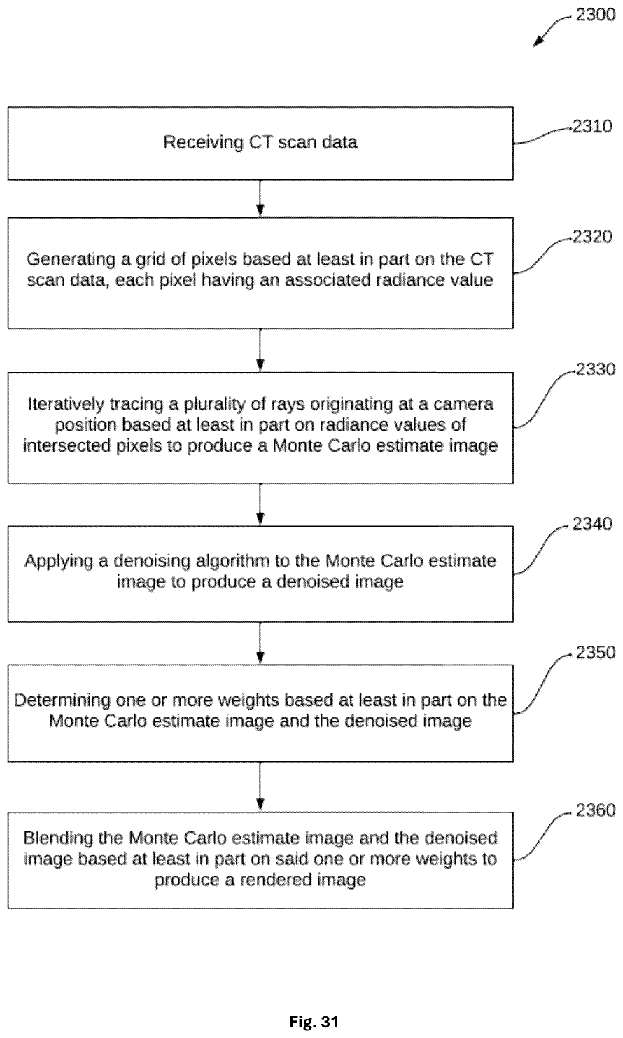

FIG. 31 is a flowchart of a method (2300) for denoising images rendered from scan data acquired by computed tomography (CT). The method includes receiving CT scan data (2310) and generating a grid of pixels, based at least in part on the CT scan data, each pixel having an associated radiance value (2320). The method further includes iteratively tracing a plurality of rays originating at a camera position based at least in part on radiance values of intersected pixels to produce a Monte Carlo estimate image (2330). The method further includes applying a denoising algorithm to the Monte Carlo estimate image to produce a denoised image (2340) and determining one or more weights for based at least in part on the Monte Carlo estimate image and the denoised image (2350). The method further includes blending the Monte Carlo estimate image and the denoised image based at least in part on said one or more weights to produce a rendered image.

### Advanced Radiological Imaging System

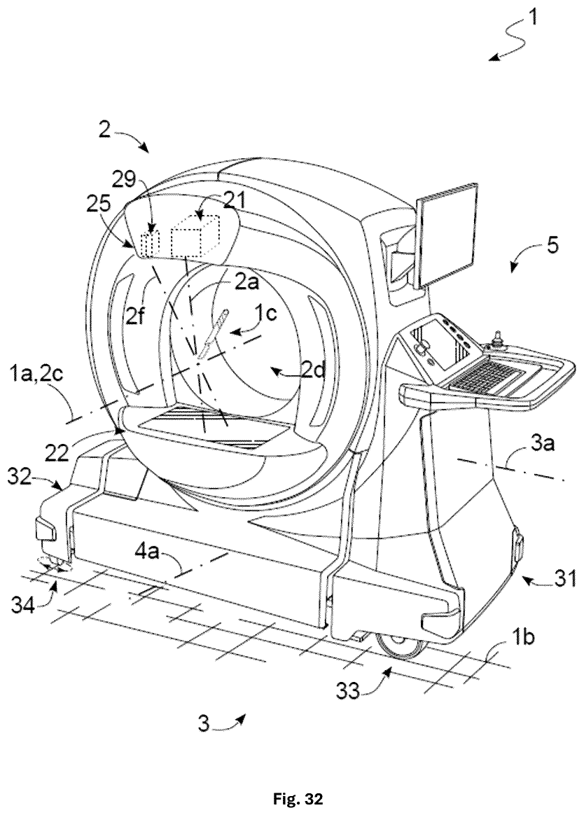

FIG. 32 depicts an embodiment of an advanced radiological imaging system 1 for use with the disclosed techniques for denoising images rendered from scan data acquired by computed tomography. The imaging system 1 is capable of conducting both 2D and 3D radiological acquisitions and tomographic scans to generate images of targeted anatomical regions of interest. The system 1 includes a gantry 2 that houses an imaging source 21 and a detector 22 that work in tandem to generate imaging data. The gantry can assume a C-shaped configuration or a closed O-shaped structure and defines a scanning zone 2d where the patient or portion of interest is positioned during imaging. The gantry supports rotational movements around a rotation axis and axial translation along a translation axis to allow for multi-slice (or stack) acquisitions needed in tomographic imaging. The system 1 operates on a platform with a support structure 3, which includes first and second columns 31, 32. The structure allows the gantry 2 to be distanced from the support surface 1b to accommodate various patient support surfaces, such as radiological beds.

A control unit (e.g., computer system 5) may comprise a graphics processing unit (GPU) in communication with GPU memory and a central processing unit (CPU) in communication with system memory, computer storage, and the GPU. The computer storage stores code which is executed by the CPU and/or the GPU to perform the techniques disclosed herein and to manage and automate imaging operations, including beam emission from the source 21, image acquisition by the detector 22, and scanner positioning via the gantry 2. It also incorporates data processing capabilities for controlling movements, managing imaging parameters, and associating positional data with acquired images. The computer system 5 includes an interface which enables the operator to control imaging parameters, monitor real-time operations, view reconstructed images, and interact a guidance systems during interventional planning. The interface can also display radiological images, composite images, and optical images for seamless interpretation.

In embodiments, during imaging operations, the system supports multi-slice acquisitions where the gantry 2 translates along the translation axis to capture multiple overlapping images of extended anatomical regions. These acquisitions are automatically stitched together to create comprehensive 3D or stacked 2D renderings.

The foregoing detailed description has presented various implementations of the devices and/or processes through the use of block diagrams, schematics, and illustrative examples. As such block diagrams, schematics, and examples include one or more functions and/or operations, it should be understood by those skilled in the art that each function and/or operation within these block diagrams, flowcharts, or examples can be implemented individually and/or collectively by employing a wide range of hardware, software, firmware, or any combination thereof. It should also be recognized by those skilled in the art that the methods or algorithms described herein may incorporate additional steps, may exclude some steps, and/or may execute steps in an order different from the one specified. The various implementations described above can be combined to create additional implementations.

These modifications and other changes can be made to the implementations in light of the above-detailed description. Generally, the terms used in the following claims should not be construed to limit the claims to the specific implementations disclosed in the specification and the claims. Instead, they should be interpreted to encompass all possible implementations, along with the full scope of equivalents to which such claims are entitled. Consequently, the claims are not limited by the disclosure but are intended to cover all possible implementations within their scope.

What is claimed is:

1. A method of denoising images rendered from scan data acquired by computed tomography (CT), comprising:
receiving CT scan data;
generating a grid of pixels based at least in part on the CT scan data, each pixel having an associated radiance value;
iteratively tracing a plurality of rays originating at a camera position based at least in part on radiance values of intersected pixels to produce a Monte Carlo estimate image;
applying a denoising algorithm to the Monte Carlo estimate image to produce a denoised image;
determining one or more weights based at least in part on the Monte Carlo estimate image and the denoised image; and
blending the Monte Carlo estimate image and the denoised image based at least in part on said one or more weights to produce a rendered image.

2. The method of claim 1, further comprising outputting the rendered image on a display.

3. The method of claim 1, wherein said determining weights comprises:
determining a first value of mean variance of the Monte Carlo estimate image in a first iteration;
determining a second value of mean variance of the Monte Carlo estimate image in a second iteration;
performing a linear regression based at least in part on said first value of mean variance and said second value of mean variance to determine a convergence relationship; and
determining said one or more weights based at least in part on the convergence relationship.

4. The method of claim 3, wherein:
said first value of mean variance of the Monte Carlo estimate image is a mean variance in a current iteration, and
said second value of mean variance of the Monte Carlo estimate image is a mean variance in an earlier iteration.

5. The method of claim 4, wherein said second value of mean variance of the Monte Carlo estimate image is estimated from said first value of mean variance of the Monte Carlo estimate image.

6. The method of claim 1, wherein said iterative tracing comprises:
(a) determining and storing a color value for each scattering ray in a first target texture based at least in part on a respective existing stored color value and a density value of a respective intersected pixel;
(b) determining and storing a position value for each scattering ray in a second target texture based at least in part on a position of the respective intersected pixel;
(c) determining and storing a scatter direction for each scattering ray in a third target texture based at least in part on the density value of the respective intersected pixel;
(d) filling a current frame buffer based at least in part on the first target texture and a previous frame buffer; and
(e) displaying the current frame buffer; and
iteratively repeating (a)-(e) until the stopping condition is met for all of the rays of the plurality of rays.

7. The method of claim 6, further comprising determining an initial origin and direction for each ray of the plurality of rays based at least in part on the camera position.

8. The method of claim 6, wherein, in an iteration, when a scattering ray does not intersect a pixel or a light source, a stopping condition of the scattering ray is met and a zero-length vector is stored in the third target texture.

9. The method of claim 6, wherein, in an iteration, when a scattering ray intersects a light source, a stopping condition of the scattering ray is met and the respective existing stored color value is attenuated based at least in part on the color of the intersected light source.

10. The method of claim 6, wherein, in an iteration, when a scattering ray exceeds a scatter number limit, a stopping condition of the scattering ray is met and the respective existing stored color value is set to zero.

11. A system for denoising images rendered from scan data acquired by computed tomography (CT), comprising:
a graphics processing unit (GPU) in communication with GPU memory;
a central processing unit (CPU) in communication with system memory, computer storage, and the GPU,
the computer storage storing code which is executed by the CPU and/or the GPU to perform a method comprising:
receiving CT scan data;
generating a grid of pixels based at least in part on the CT scan data, each pixel having an associated radiance value;
iteratively tracing a plurality of rays originating at a camera position based at least in part on radiance values of intersected pixels to produce a Monte Carlo estimate image;
applying a denoising algorithm to the Monte Carlo estimate image to produce a denoised image;
determining one or more weights based at least in part on the Monte Carlo estimate image and the denoised image; and
blending the Monte Carlo estimate image and the denoised image based at least in part on said one or more weights to produce a rendered image.

12. The system of claim 11, further comprising a display, and code which is executed by the CPU and/or the GPU to perform outputting the rendered image on the display.

13. The system of claim 11, further comprising code which is executed by the CPU and/or the GPU to perform, in said determining weights:
determining a first value of mean variance of the Monte Carlo estimate image in a first iteration;
determining a second value of mean variance of the Monte Carlo estimate image in a second iteration;
performing a linear regression based at least in part on said first value of mean variance and said second value of mean variance to determine a convergence relationship; and
determining said one or more weights based at least in part on the convergence relationship.

14. The system of claim 13, wherein:
said first value of mean variance of the Monte Carlo estimate image is a mean variance in a current iteration, and
said second value of mean variance of the Monte Carlo estimate image is a mean variance in an earlier iteration.

15. The system of claim 14, wherein said second value of mean variance of the Monte Carlo estimate image is estimated from said first value of mean variance of the Monte Carlo estimate image.

16. A computer-readable medium storing code for denoising images rendered from scan data acquired by computed tomography (CT), wherein the code, when executed by a central processing unit (CPU) and/or a graphics processing unit (GPU), performs a method comprising:
receiving CT scan data;
generating a grid of pixels based at least in part on the CT scan data, each pixel having an associated radiance value;
iteratively tracing a plurality of rays originating at a camera position based at least in part on radiance values of intersected pixels to produce a Monte Carlo estimate image;
applying a denoising algorithm to the Monte Carlo estimate image to produce a denoised image;
determining one or more weights based at least in part on the Monte Carlo estimate image and the denoised image; and
blending the Monte Carlo estimate image and the denoised image based at least in part on said one or more weights to produce a rendered image.

17. The computer-readable medium of claim 16, further comprising code for outputting the rendered image on a display.

18. The computer-readable medium of claim 16, wherein said determining weights comprises:
determining a first value of mean variance of the Monte Carlo estimate image in a first iteration;
determining a second value of mean variance of the Monte Carlo estimate image in a second iteration;
performing a linear regression based at least in part on said first value of mean variance and said second value of mean variance to determine a convergence relationship; and
determining said one or more weights based at least in part on the convergence relationship.

19. The computer-readable medium of claim 18, wherein:
said first value of mean variance of the Monte Carlo estimate image is a mean variance in a current iteration, and
said second value of mean variance of the Monte Carlo estimate image is a mean variance in an earlier iteration.

20. The computer-readable medium of claim 19, wherein said second value of mean variance of the Monte Carlo estimate image is estimated from said first value of mean variance of the Monte Carlo estimate image.
